(19) United States
(12) Patent Application Publication
(10) Pub. No.: US 2024/0400979 A1
(43) Pub. Date: Dec. 5, 2024

(54) CHICKPEA MICROCARRIERS

(71) Applicant: Upside Foods, Inc., Berkeley, CA (US)

(72) Inventors: Yulong Liu, Emeryville, CA (US); Suzanne Burke Emerson, San Francisco, CA (US); Michaela Jane Walker, Emeryville, CA (US)

(21) Appl. No.: 18/732,280

(22) Filed: Jun. 3, 2024

Related U.S. Application Data
(60) Provisional application No. 63/505,989, filed on Jun. 2, 2023.

(51) Int. Cl. C12N 5/00; A23J 3/14; A23J 3/22; A23L 11/00

(52) U.S. Cl. CPC C12N 5/0075; A23J 3/14; A23J 3/227; A23L 11/05; C12N 2531/00; C12N 2533/90

(57) ABSTRACT

This disclosure describes methods for growing cells on chickpea microcarriers as part of a process of creating a comestible food product. Generally, the disclosed method comprises adding chickpea microcarriers to cell culture media in a vessel. The chickpea microcarriers are mixed with non-human cells in the cell culture media. The chickpea microcarriers and non-human cells are mixed to adhere and grow the non-human cells on the surface of the chickpea microcarriers. After mixing, the non-human cells adhered to the surface of the chickpea microcarriers forms a textured cell tissue. The textured cell tissue can be harvested from the vessel and formed into a comestible food product.

### CROSS-REFERENCE TO RELATED APPLICATIONS

This application claims priority to and the benefit of U.S. Provisional Patent Application No. 63/505,989, titled, “CHICKPEA MICROCARRIERS,” filed on Jun. 2, 2023, which is incorporated herein by reference in its entirety.

### BACKGROUND

As the world's population continues to grow, cell-based or cultured food products (e.g., cell-based meat products) for consumption have emerged as an attractive alternative (or supplement) to conventional meat from animals. For instance, cell-based, cultivated, or cultured meat represents a technology that could address the specific dietary needs of humans. Cell-based food products can be prepared from a combination of cultivated adherent and suspension cells derived from a non-human animal. Because the cells for cell-based meat are made in a food cultivation facility, cell masses are often formed and shaped to mimic familiar forms of conventional meat.

In addition to addressing dietary needs, cell-based food products help alleviate several drawbacks linked to conventional food products for humans, livestock, and the environment. For instance, conventional meat production involves controversial practices associated with animal husbandry, slaughter, and harvesting. Other drawbacks associated with harvested or slaughtered meat production include low conversion of caloric input to edible nutrients, microbial contamination of the product, emergence and propagation of veterinary and zoonotic diseases, relative natural resource requirements, and resultant industrial pollutants, such as greenhouse gas emissions and nitrogen waste streams.

Despite advances in creating cell-based food products, existing methods or systems for cultivating and processing cell-based food products face several shortcomings, such as challenges or failures to mimic the textures and flavors of slaughtered or harvested meat. In particular, some existing methods or systems often produce cell-based food products with undesirable textures. For instance, certain systems often grow cells in a pure single cell suspension. While the cells grow in the pure single cell suspension, they are typically unable to form muscle fibers or other multicellular structures. This inability to form multicellular structures can cause cell-based food products comprised of the grown cells to be too soft or suffer from other textural drawbacks.

In addition to poor texture, existing methods or systems of forming cell-based or cultured meat often utilize inefficient scaffolds to improve the textures of cell-based food products. For example, some existing scaffolds are large, unitary structures that require surfaces with specific geometric properties or surface coatings to adhere to cells. In certain cases, scaffolds must be removed from the adhered cells prior to forming the cell-based food product. Such removal requires further processing and can decrease the collection of cell deposited protein and/or disrupt the multicellular structures or cell morphologies that improve cell-based food product texture. In some existing systems, scaffolds must use specialized vessels that anchor the scaffolds while the non-human cells grow and differentiate.

Moreover, several existing methods or systems of forming cell-based or cultured meat utilize inflexible or undesirable cell culture media compositions. For example, many existing systems must use animal serum (e.g., Fetal Bovine Serum) in cell culture media to promote the growth and differentiation of non-human cells. The cost and sustainability of utilizing animal serum make these approaches suboptimal. Thus, many existing systems are limited to and by certain cell media compositions.

These, along with additional problems and issues exist in existing methods for cultivating cell-based food products.

### BRIEF SUMMARY

This disclosure generally describes methods for growing cells on chickpea microcarriers as part of a process of creating cell-based food products (e.g., cell-based meat products). For example, the disclosed method can include adding chickpea microcarriers comprising ground and textured chickpea protein to cell culture media in a vessel for cell suspension, such as a bioreactor or cultivator. The chickpea microcarriers can further be mixed with non-human cells in the cell culture media. In some cases, for instance, the chickpea microcarriers and non-human cells are mixed within the cell culture media at different rates during different time periods. During mixing, the non-human cells can adhere to (and grow on) the surface of the chickpea microcarriers. After a mixing period, a growth period, a maturation period, a differentiation period, or some combination thereof, the textured cell tissue, made up of the chickpea microcarriers and adhered cells, can be harvested from the vessel and formed into a comestible food product.

Additional features and advantages of one or more embodiments of the present disclosure will be set forth in the description that follows.

### BRIEF DESCRIPTION OF THE DRAWINGS

Various embodiments will be described and explained with additional specificity and detail through the use of the accompanying drawings, which are summarized below.

FIG. 1 illustrates an overview diagram of forming a textured cell tissue into a comestible food product in accordance with one or more embodiments of the present disclosure.

FIG. 2 illustrates forming a chickpea microcarrier in accordance with one or more embodiments of the present disclosure.

FIGS. 3A-3B illustrate adding and mixing chickpea microcarriers, non-human cells, and cell culture media in accordance with one or more embodiments of the present disclosure.

FIG. 4 illustrates non-human cells adhering to the surface of a microcarrier and cell differentiation in accordance with one or more embodiments of the present disclosure.

FIG. 5 illustrates an example series of acts for harvesting textured cell tissue and forming a comestible food product in accordance with one or more implementations of the present disclosure.

FIGS. 6A-6B illustrate the adherence of non-human cells to textured chickpea protein, and existing microcarrier beads in serum-containing and serum free cell culture media in accordance with one or more embodiments of the present disclosure.

FIGS. 7A-7B illustrate various seeding densities of non-human cells at different time periods in accordance with one or more embodiments of the present disclosure.

FIGS. 8A-8B illustrate images of textured cell tissue comprising non-human cells adhered to chickpea microcarriers at different mixing speeds during an initial time period and subsequent time period in accordance with one or more embodiments of the present disclosure.

FIG. 9 illustrates how a lower initial mixing speed and subsequent mixing speed can lead to aggregates of non-human cells in accordance with one or more embodiments of the present disclosure.

FIG. 10 illustrates an amount of protein (e.g., protein per culture volume) of the textured cell tissue with various media additives in accordance with one or more embodiments of the present disclosure.

FIG. 11 illustrates a series of acts for forming a comestible food product in accordance with one or more embodiments of the present disclosure.

FIG. 12 illustrates a series of acts for forming chickpea microcarriers in accordance with one or more embodiments.

FIGS. 13A-13D illustrate an overview diagram of growing and processing different types of cells in accordance with one or more embodiments of the present disclosure.

### DETAILED DESCRIPTION

This disclosure describes one or more implementations of a method for growing adhering, and/or maturing non-human cells on a chickpea microcarrier as part of a process of creating cell-based food products. In one or more embodiments, the disclosed method comprises adding chickpea microcarriers comprising textured chickpea protein to a cell culture media. The disclosed method further comprises mixing the chickpea microcarriers with non-human cells in the cell culture media. In some cases, the chickpea microcarriers, non-human cells, and cell culture media are mixed at different rates, such as by increasing a mixing rate over a time period or by using different mixing rates at different time periods. The disclosed method further comprises harvesting textured cell tissue comprising the chickpea microcarriers and non-human cells adhering to a surface of the chickpea microcarriers. Such harvested textured cell tissue can comprise grown, matured, and/or differentiated non-human cells. In some embodiments, the disclosed method forms the harvested textured cell tissue into a comestible food product (e.g., a comestible meat product).

As indicated above, the disclosed method includes forming chickpea microcarriers. In one or more embodiments, the disclosed method (i) extracts chickpea protein from chickpeas and (ii) adds chickpea flour to the extracted chickpea proteins to create a textured chickpea protein. Either before or after adding chickpea flour, in certain embodiments, the disclosed method micronizes or otherwise reduces the textured chickpea protein into chickpea microcarriers by grinding the textured chickpea protein to a particular granularity or size. In addition to size-reducing, in certain embodiments, the disclosed method ensures that the chickpea microcarrier is the particular size by filtering the chickpea microcarrier through a sieve or other filter. Once the chickpea microcarrier reaches the particular size, in one or more embodiments, the disclosed method sterilizes the chickpea microcarrier.

As mentioned, the disclosed method comprises adding chickpea microcarriers to cell culture media within a vessel. In some embodiments, the cell culture media is free of animal serums (e.g., Fetal Bovine Serum “FBS”). In one or more embodiments, the cell culture media contains non-human cells before adding the chickpea microcarriers. As depicted and described further below, in other embodiments, the disclosed methods comprise adding both non-human cells and chickpea microcarriers to the cell culture media at the same time.

The disclosed method further includes mixing the non-human cells with the chickpea microcarriers within the cell culture media. In particular, the disclosed method can utilize various agitation methods to enable adherence of the non-human cells to the surface of the chickpea microcarriers. For instance, in some cases, an agitation method is required for non-human cells and the chickpea microcarriers to stay in suspension, but gentle enough to allow the non-human cells to attach and stay attached to the chickpea microcarrier. In general, agitation is antithetical to adherence between chickpea microcarriers and non-human cells, so a proper balance must be achieved such that agitation is strong enough to maintain the coated microcarriers in suspension, but weak enough to allow the non-human cells to remain adhered to the chickpea microcarriers. The combination of proper agitation and cell adherence to the chickpea microcarriers in cell culture media facilitates or creates an environment for cell differentiation over time. To illustrate, in some cases, the disclosed method sets an initial mixing speed (e.g., rate) of 60 rotations per minute (“RPM”) (or other mixing speed suitable for a scale of the equipment) to promote an environment in which the cells can adhere to the textured chickpea microcarriers. After a period of time, the disclosed method increases the initial mixing speed to 100 RPMs (or other increased mixing speed suitable for the scale of the equipment), which further fosters an environment in which the non-human cells grow, differentiate, exhibit a certain phenotype or some combination thereof. In some embodiments, the disclosed method maintains the increased rate until the non-human cells are ready to harvest.

As mentioned above, by adhering to the chickpea microcarriers, the non-human cells and the chickpea microcarriers together form a textured cell tissue. In some cases, the textured cell tissue is harvested by removing the textured cell tissue from the cell culture media. For instance, the disclosed method concentrates the textured cell tissue by draining the cell culture media through a sieve. In some cases, the disclosed method reduces a moisture content of the textured cell tissue by additionally or alternatively pressing the textured cell tissue. In other cases, the disclosed method reduces the moisture content by centrifuging the textured cell tissue.

After harvesting, the disclosed method can form the harvested textured cell tissue into a comestible food product. For instance, the textured cell tissue may be arranged within a mold having a shape of a target cut of meat to mimic the muscular architecture/shape of the target cut of meat. In particular, the mold may comprise grooves that cause the textured cell tissue to have a surface texture that mimics the surface texture of the target cut of meat.

The disclosed method provides several benefits relative to existing methods for growing cell-based meats. In particular, the disclosed method provides an improved adherent surface for non-human cells and, in some cases, facilitates the production of additional extracellular matrix (“ECM”). By facilitating the production of the ECM, the disclosed method provides a substrate and environment within which non-human cells can differentiate to a cell type and/or produce a protein that, when embedded with the chickpea microcarriers in a product, exhibits a desirable texture. As part of improving texture relative to existing cell-based food products—because the adherence of the non-human cells to chickpea microcarriers—the disclosed method produces, and thus, collects more ECM than existing methods and provides a structure that forms a more robust three-dimensional (3D) tissue. Accordingly, the disclosed method forms cell-based food products (e.g., cell-based meat products) having improved texture, structure, and shape relative to existing methods.

In addition to increasing the growth of non-human cells exhibiting an improved texture, the disclosed method improves the efficiency of growing cells and harvesting textured cell tissue. In particular, because chickpea microcarriers are edible, stable, textured, and hypoallergenic, they likewise expedite the process of growing differentiated cells and forming a cell-based food product that includes not only the cells but also suitable microcarriers that do not require removal. For instance, it is hypothesized that due to their texture, the chickpea microcarriers do not require lengthy processing to ensure adherence between them and the non-human cells. Moreover, the disclosed method reduces the degree of processing for harvested cell tissue because the chickpea microcarriers remain in the harvested cell tissue.

Beyond efficient cell growth, the disclosed method expands the type of cell culture media that can be used to effectively grow non-human cells for cell-based food products. For example, the disclosed method creates an environment in which non-human cells adhere to the chickpea microcarriers in animal-serum-free media. By eliminating animal serum, the disclosed method can utilize various cell culture media formulations without the limits of existing, animal-serum-based methods.

As described further below, in contrast to chickpea microcarriers, soy and pea textured microcarriers lack the media range-media range referring to, for example, amenability to different media types and formulation in which the microcarriers retain of function—and adherence of chickpea microcarriers. For example, after mixing the non-human cells and the soy and pea textured microcarriers in the cell culture media, the non-human cells showed minimal attachment to soy and pea textured microcarriers in cell culture media without FBS. The soy and pea textured microcarriers lack media range relative to chickpea microcarriers because the soy and pea textured microcarriers require cell culture media containing animal serum (e.g., FBS) to provide an environment where non-human cells can adhere to a surface of the soy and pea textured microcarriers in a suspension culture environment. Additionally, soy and pea textured microcarriers lack the adherence capabilities of chickpea microcarriers. For example, non-human cells either do not attach to or have minimal attachment to soy and pea textured microcarriers in serum free cell culture media. Conversely, non-human cells attach to chickpea microcarriers in serum free cell culture media, which allows the non-human cells to grow, differentiate, and exhibit a certain phenotype, or some combination thereof on the chickpea microcarrier. The ability of cells to attach to chickpeas but not to soy and pea under serum free conditions was surprising to the inventors.

As illustrated by the foregoing discussion, the present disclosure utilizes a variety of terms to describe features and advantages of the disclosed method. Additional detail is now provided regarding the meaning of such terms. As used herein, the term “cells” (or “non-human cells”) refers to cells that form food products (e.g., meat products). Generally, non-human cells may comprise at least one of muscle cells, muscle progenitor cells, or muscle support cells. In particular, non-human cells may comprise different cell types, such as one or more of myoblasts, mesoangioblasts, myofibroblasts, mesenchymal stem cells, hepatocytes, fibroblasts, pericytes, adipocytes, epithelial cells, chondrocytes, osteoblasts, osteoclasts, pluripotent cells, somatic stem cells, endothelial cells, embryonic stem cells, induced pluripotent stem cells, or other similar cell types. Furthermore, cells may comprise different types of progenitor cells, including myogenic progeny and progenitors, adipogenic progeny or progenitors, mesenchymal progeny or progenitors, or other types of progenitor cells. When seeding, in some embodiments, the disclosed method includes seeding telomerase reverse transcriptase (TERT) immortalized chicken fibroblasts or other immortalized cells, spontaneously immortalized or otherwise.

As used herein, the term “textured cell tissue” refers to a tissue or mass comprising non-human cells and chickpea microcarriers. For instance, the textured cell tissue can include cells of cultivated meat (e.g., non-human cells) adhered to chickpea microcarriers which are gathered into a collective mass. Such a textured cell tissue may nevertheless be raw or uncooked. In some embodiments, the textured cell tissue is comestible. Additionally, a textured cell tissue may include grown non-human cells that have been nourished by a growth medium (e.g., cell culture media) to grow during a formation period within a cultivator. In some embodiments, a textured cell tissue may include matured or differentiated non-human cells that may have been exposed to differentiation media and/or conditions favoring differentiation to form structures such as, for example, myotubes. In some examples, textured cell tissue is grown from mixing non-human cells and chickpea microcarriers floating and/or suspended in liquid or gel in a suspension reactor/cultivator.

As used herein, the term “microcarrier” refers to a spherical or irregular-shaped and distinct support matrix that adheres or attaches to cells within a cultivator. A microcarrier can be a discrete, free-floating granule (e.g., unit) in a suspension. During use, a microcarrier is placed within a cell cultivator and suspended within a cell culture media but the microcarrier does not otherwise maintain a fixed position or orientation relative to a cell cultivator. Microcarriers may comprise different materials. In some instances, microcarriers can be edible or inedible. For example, microcarriers can comprise edible materials, such as chickpea protein, soybean protein, pea protein, polysaccharides, polypeptides, lipids, pectin, gelatin, dextran, or cellulose. In other cases, microcarriers can comprise inedible materials, such as, glass, plastic, dextran, or polystyrene. While this disclosure describes chickpea-protein-based microcarriers that are edible, in some embodiments, such microcarriers may include other edible components in addition to chickpea protein.

As used herein, the term “differentiation” refers to a process by which a cell changes from an initial cell type to a different cell type with a more specialized form and/or role. For instance, differentiated cells have specific structures and functions. Differentiation can be used so that cells mature to exhibit a certain phenotype characteristic of cells, such as, myoblasts, mesoangioblasts, myofibroblasts, mesenchymal stem cells, hepatocytes, fibroblasts, pericytes, adipocytes, epithelial cells, chondrocytes, osteoblasts, osteoclasts, pluripotent cells, somatic stem cells, endothelial cells, embryonic stem cells, induced pluripotent stem cells, or myogenic stem cells.

As used herein, the term “extracellular matrix” (or “ECM”) refers to a three-dimensional network that supports cell growth, adhesion, and differentiation. For instance, an ECM is an interwoven mesh comprising of glycosaminoglycans (e.g., hyaluronan), glycoproteins (e.g., fibronectin, laminin, etc.), and/or fibrous proteins (e.g., collagen, elastin, etc.). The ECM is secreted by cells and forms a network surrounding the cells that helps the cells communicate with one another. For example, the ECM can refer to the structure adhering cells to each other. In some embodiments, the ECM adheres to a microcarrier. Additionally, the ECM helps regulate cell differentiation. For example, based on the rigidity of the ECM, the non-human cells may develop into different cell types.

As used herein, the term “cell culture media” refers to a media (e.g., liquid, gel, etc.) that provides nutrients to cells and supports cell growth, cell differentiation, or both. In some cases, cell culture media assists in regulating environmental conditions. Cell culture media can be natural (e.g., extracted from animal tissue or animal body fluids) or synthetic (e.g., combinations of organic or inorganic compounds). For instance, cell culture media can comprise any combination of amino acids, vitamins, carbohydrates, inorganic salts, minerals, supplements, glucose, serums, or hormones. In certain cases, cell culture media can be animal component free (“ACF”). For instance, ACF cell culture media does not contain FBS.

As used herein, the term “harvest” or “harvesting” refers to a process of removing cells from a controlled environment. For example, harvesting refers to the process of removing textured cell tissue from a controlled environment by draining a portion of the cell culture media. In one or more implementations, harvesting comprises reducing the moisture content of the textured cell tissue by pressing the textured cell tissue.

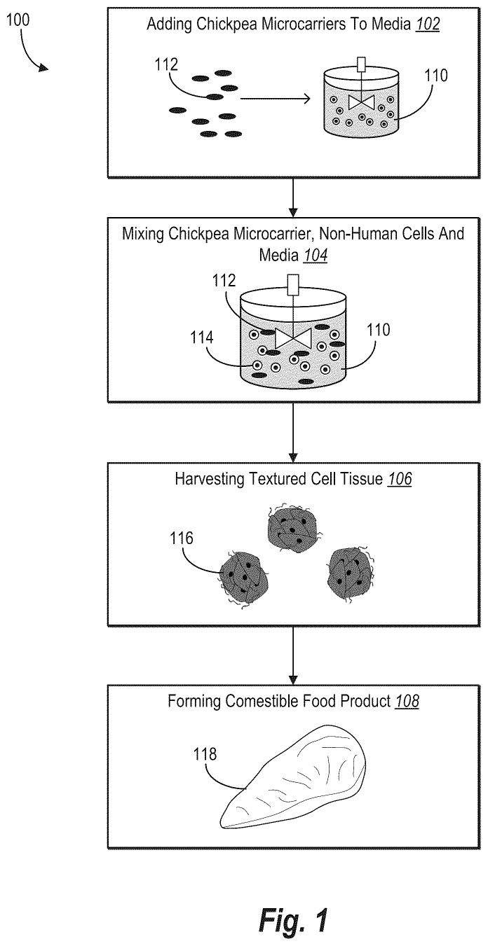

Additional detail will now be provided regarding disclosed methods in relation to illustrative figures portraying example embodiments and implementations of the disclosed methods and apparatuses. FIG. 1 illustrates an overview 100 of utilizing chickpea microcarriers to form a comestible food product in accordance with one or more embodiments.

FIG. 1 illustrates the act 102 of adding chickpea microcarriers to media. For example, the act 102 comprises adding chickpea microcarriers 112 to cell culture media 110 within a bioreactor. In some embodiments, the cell culture media is serum free. For example, the cell culture media does not contain any animal serums (e.g., newborn calf serum, horse serum, etc.), is not comprised of bovine sourced sera (e.g., FBS), or both.

In some implementations, the act 102 of adding chickpea microcarriers to cell culture media includes adding the chickpea microcarriers at the same time as adding the non-human cells. By adding the chickpea microcarriers and the non-human cells at the same time, the disclosed method allows the non-human cells to grow, mature, differentiate, or some combination thereof while attached to the surface of the chickpea microcarrier.

By contrast, in one or more embodiments, the act 102 of adding chickpea microcarriers to cell culture media includes adding the chickpea microcarriers to cell culture media already containing non-human cells. For instance, non-human cells may be in the cell culture media as single cell suspension to grow the non-human cells. In some cases, the non-human cells grow to high density prior to adding the chickpea microcarriers. In some embodiments, the non-human cells are grown to high density, subsequently chickpea microcarriers are added, and subsequently the non-human cells are further grown, matured, differentiated, or some combination thereof.

In some cases, the act 102 includes adding dry chickpea microcarriers to the cell culture media. For instance, the disclosed method can transfer dry chickpea microcarriers from a container vessel to a bioreactor that mixes together the chickpea microcarriers, the non-human cells, and the cell culture media. In addition or in the alternative to drying chickpea microcarriers prior to adding them to cell culture media, in some embodiments, the disclosed method hydrates the chickpea microcarriers prior to adding them to the cell culture media. For instance, the disclosed method hydrates the chickpea microcarriers by adding cell culture media to dry chickpeas in an aseptic container. In some cases, the disclosed method transfers the hydrated chickpea microcarriers to a larger bioreactor that mixes the chickpea microcarriers, non-human cells, and cell culture media. As explained further below, in some embodiments, the chickpea microcarriers are coated with an adhesive material or other coating prior to adding to the cell culture media.

FIG. 1 further illustrates the act 104 of mixing chickpea microcarriers, non-human cells, and media. In particular, the act 104 comprises agitating the cell culture media 110 within the bioreactor. As illustrated, the act 104 evenly distributes the chickpea microcarriers 112 and non-human cells 114 in the cell culture media 110. Generally, even distribution encourages the non-human cells to adhere to and/or evenly coat a surface of the chickpea microcarriers. Moreover, a proper mixing rate (e.g., degree of agitation) and even distribution enables sufficient gas and nutrient diffusion, integration of feeds or other additions, and stable temperatures throughout the mixing process.

In one example, the act 104 comprises mixing the chickpea microcarriers, non-human cells, and cell culture media at different rates and/or intensities. For example, the act 104 includes an initial mixing speed during an initial time period where the non-human cells adhere to the chickpea microcarrier. In some embodiments, after the initial time period that is customized for cell attachment to the chickpea microcarriers, the disclosed method increases the initial mixing speed to allow increased nutrient mixing and growth relative to the initial time period until the disclosed method harvests the non-human cells adhered to the chickpea microcarriers.

In some embodiments, the act 104 facilitates and helps create an environment for cell differentiation. In particular, during mixing, the non-human cells differentiate to exhibit a certain phenotype. For instance, while the cells adhere to the chickpea microcarrier, the cells contact each other and exchange biochemical signals. In some cases, the cell-to-cell contact allows the cells to fuse together to form myoblasts and myotubes on a surface of the chickpea microcarrier.

As further illustrated in FIG. 1, the disclosed method includes the act 106 of harvesting textured cell tissue. The act 106 comprises separating textured cell tissue 116, comprising the adhered non-human cells to the chickpea microcarriers, from the cell culture media. For instance, the act 106 includes draining the cell culture media from the bioreactor through a sieve. In some embodiments, the act 106 includes separating the textured cell tissue from the cell culture media by centrifuging the textured cell tissue.

In addition to harvesting, in certain embodiments, the disclosed method further comprises washing the textured cell tissue. For instance, the disclosed method includes rinsing the textured cell tissue with a wash buffer. In some implementations, the disclosed method rinses the textured cell tissue over a filter. In certain embodiments, the disclosed method further comprises drying the textured cell tissue. For example, the disclosed method comprises reducing the moisture content of the textured cell tissue by pressing the textured cell tissue on a cell press.

As further illustrated in FIG. 1, the disclosed method includes the act 108 of forming a comestible food product. For instance, the textured cell tissue may be placed in a mold (e.g., hollow container) to give shape to the textured cell tissue and to form a comestible food product 118. In some embodiments, the act 108 further comprises arranging and/or layering the textured cell tissue to mimic a target or desired cut of meat (e.g., chicken breast, sirloin steak, fish filet, etc.).

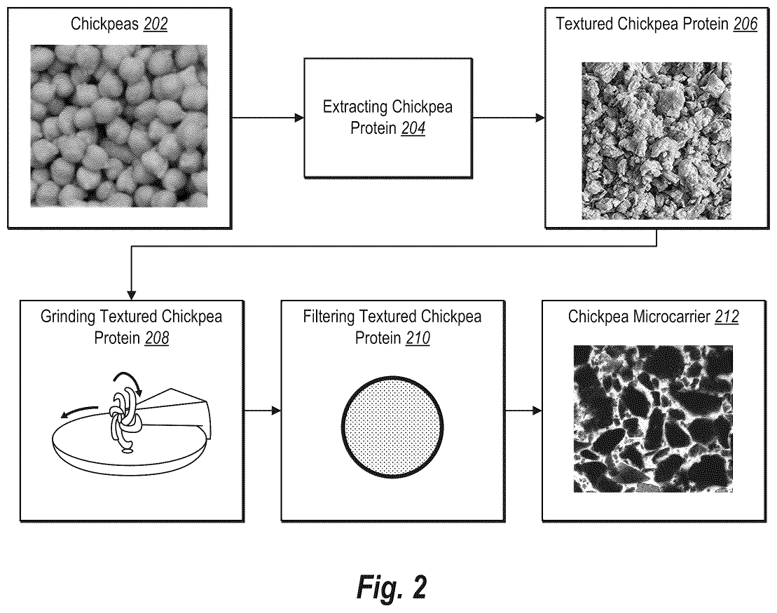

As previously mentioned, in some implementations, the disclosed method comprises forming chickpea microcarriers. FIG. 2 illustrates an example method of forming chickpea microcarriers in accordance with one or more embodiments. As shown in FIG. 2, the disclosed method starts with obtaining chickpeas 202 (e.g., garbanzo beans). As further illustrated in FIG. 2, the disclosed method includes an act 204 of extracting and/or isolating chickpea protein from chickpeas 202. For example, the chickpea protein may be extracted/isolated with acid-base solvents, salting out methods, and/or enzymatic hydrolysis methods. In some embodiments, the isolated chickpea protein is dried prior to grinding. For instance, the disclosed method may include drying the chickpea protein with heat or freeze-dry techniques prior to passing the chickpea protein through a grinder or a mill.

As shown in FIG. 2, once chickpea protein has been extracted, the disclosed method can add chickpea flour to the chickpea protein to form a textured chickpea protein 206. As further illustrated in FIG. 2, the disclosed method includes the act 208 of grinding the textured chickpea protein into discrete granules that are provided as chickpea microcarriers. In particular, the disclosed method forms the chickpea microcarriers by micronizing the textured chickpea proteins. For instance, the disclosed method grinds/mills the textured chickpea protein to a particular size, texture, or granularity. For instance, the disclosed method grinds the chickpea proteins to form chickpea microcarriers ranging from 100-300 microns (μm). By contrast, in other embodiments, the disclosed method grinds the chickpea proteins to form chickpea microcarriers ranging from 60-80 μm.

In some embodiments, the disclosed method utilizes a burr grinder, a mill, or a biopsy punch that crushes or otherwise reduces the size of the textured chickpea protein granules by force between two surfaces. In other embodiments, the disclosed method utilizes a blade grinder to cut the textured chickpea protein granules into smaller units. In some embodiments, the disclosed method grinds the textured chickpea protein multiple times. For instance, the disclosed method may grind the textured chickpea protein three times before filtering the discrete granules of the textured chickpea protein. In additional embodiments, the disclosed method grinds the textured chickpea proteins for a length of time until they reach a specific granularity or size.

As further illustrated in FIG. 2, the disclosed method includes the act 210 of filtering textured chickpea proteins. In particular, the disclosed method passes the discrete granules of textured chickpea protein through a filter to collect/isolate the chickpea microcarriers. In some embodiments, the disclosed method passes the discrete granules of the textured chickpea protein through a strainer or sifter.

For example, in some embodiments, the filter, strainer, or sifter includes filtering holes of 200 μm or less in size. For example, in one or more embodiments, the disclosed method utilizes a filter comprising filtering holes that do not exceed 80 μm. Thus, in certain cases, the disclosed method filters the micronized, discrete granules of the textured chickpea protein to generate chickpea microcarriers 212 that do not exceed a certain size. For instance, the chickpea microcarriers do not exceed 150 μm or the size of chickpea microcarriers is 80 μm or less.

In some implementations, the disclosed method further comprises sanitizing the chickpea microcarriers. To illustrate, the disclosed method may sterilize the chickpea microcarriers with sterile solutions. For instance, the disclosed method sterilizes the chickpea microcarriers with a phosphate buffered saline (PBS) wash, such as antibiotic-antimycotic (“anti-anti”) PBS wash. In other embodiments, the disclosed method sterilizes the chickpea microcarriers with heat. For instance, the disclosed method places the chickpea microcarriers into an autoclave and heats them under pressure with steam for a period of time. For example, the disclosed method may place the microcarriers in the autoclave a temperature of 110-130° C. for at least 20-40 minutes under 10-20 pounds per square inch (e.g., a temperature of 121° C. for at least 30 minutes under 15 pounds per square inch). In some embodiments, the disclosed method may store the sanitized chickpea microcarriers in sanitized containers prior to adding them to the cell culture media.

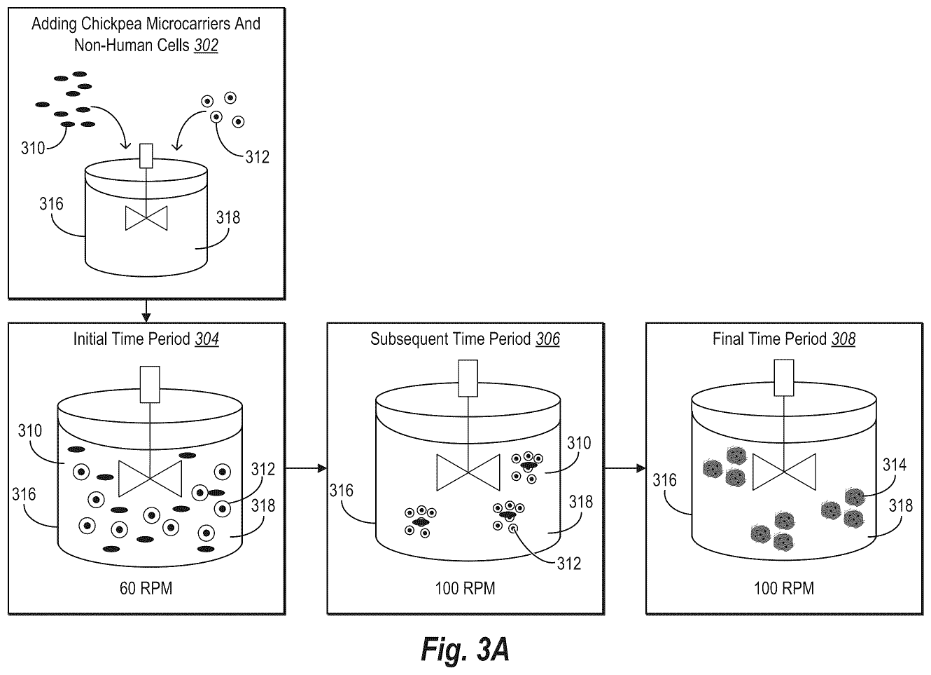

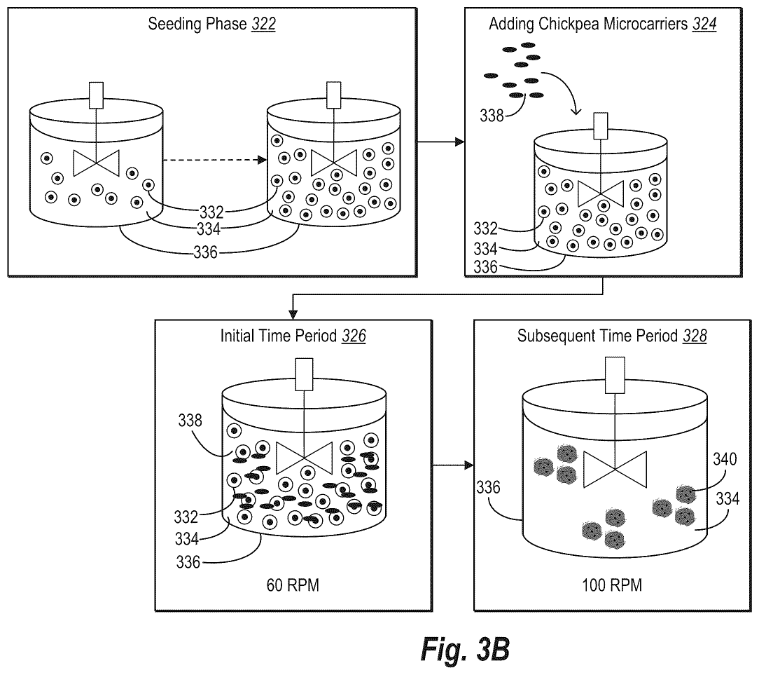

As discussed above, the disclosed method adds the chickpea microcarriers to a cell culture media and mixes the chickpea microcarriers, non-human cells, and cell culture media. FIGS. 3A-3B illustrate adding and mixing chickpea microcarriers with the non-human cells in cell culture media in accordance with one or more implementations.

Generally, the type of cell culture media affects how the non-human cells adhere to a surface. For instance, cell culture media comprising animal sourced serum enables cells to attach to several surfaces. However, animal sourced serum is expensive, and extraction of animal sourced serum raises ethical issues. In the alternative to animal sourced serum, the disclosed method can use serum-free cell culture media. For example, in some cases, the cell culture media does not contain any animal sourced serum (e.g., FBS, newborn calf serum, horse serum, etc.). In some embodiments, the media comprises suspension yellow lion (“SusYL”), a proprietary animal-component-free (ACF) media. In addition to an underlying media, in one or more cases, the cell culture media contains other reagents that feed the non-human cells.

In the alternative to serum-free media, the cell culture media can contain animal sourced serum. For example, in certain embodiments, the cell culture media contains a portion of animal sourced serum. Such cell culture media can comprise a mixture of SusYL and 10% FBS or other type of animal serum.

As further shown in FIG. 3A, the disclosed method includes the act 302 of adding chickpea microcarriers and non-human cells to the cell culture media. As illustrated, in some embodiments, the disclosed method concurrently adds chickpea microcarriers 310 and non-human cells 312 to cell culture media 318 within a bioreactor 316. In particular, the disclosed method adds enough chickpea microcarriers to optimize cell growth in a suspension. For example, the disclosed method may include depositing 2-5 grams of dry chickpea microcarriers per liter (L) of cell culture media into the bioreactor containing the cell culture media, such as 3 g/L of total volume. In one or more embodiments, the disclosed method includes drying the chickpea microcarriers through heat or freeze-drying prior to adding them to the cell culture media.

In some implementations, the disclosed method directly adds dry chickpea microcarriers 310 to the cell culture media 318. For instance, the disclosed method transfers the dry chickpea microcarriers 310 from a container to the bioreactor 316 that mixes the chickpea microcarriers 310, non-human cells 312, and cell culture media 318. To illustrate, the disclosed method can deposit the dry chickpea microcarriers 310 from an aseptic container to an enclosed bioreactor through a sterile tube connecting the enclosed container and the enclosed bioreactor.

Alternatively, in some implementations, chickpea microcarriers 310 are hydrated prior to adding them to the cell culture media 318 in the bioreactor 316. For example, the disclosed method may include adding a portion of the cell culture media 318 to the chickpea microcarriers 310 in an aseptic container. Once hydrated, the disclosed method can pump the hydrated chickpea microcarriers 310 from the aseptic container to an enclosed bioreactor containing a remaining portion of the cell culture media 318 and the non-human cells 312.

In some implementations, the chickpea microcarriers are coated with an adhesive material prior to adding the chickpea microcarriers 310 to the cell culture media 318. For instance, the disclosed method may coat the chickpea microcarriers 310 with transglutaminase and/or other bonding materials prior to adding the chickpea microcarriers 310 to the cell culture media 318. In some cases, coating the chickpea microcarriers 310 with the adhesive materials further enhances adherence, differentiation, ECM production, and allows for higher agitation rates.

As shown in FIG. 3A, the disclosed method concurrently adds the chickpea microcarriers 310 and the non-human cells 312 to the cell culture media 318 within the bioreactor 316. In particular, the disclosed method seeds the non-human cells at a low density. For example, the disclosed method seeds 200,000-300,000 non-human cells per liter of cell culture media (e.g., 250,000 non-human cells/L).

In some embodiments, prior to adding the non-human cells 312, the disclosed method prepares the non-human cells 312 for seeding. For instance, the disclosed method thaws the non-human cells 312 and, in some instances, scales up the cells to the desired density. To illustrate, the disclosed method thaws a cryovial containing frozen non-human cells 312 (e.g., chicken fibroblast, bovine fibroblast, etc.). After thawing, the disclosed method can grow the thawed non-human cells 312 in the cell culture media 318 or another growing reagent until they reach a target density (e.g., 250,000 to 30 million non-human cells/ml).

As further shown in FIG. 3A, after the disclosed method adds the chickpea microcarriers 310, the disclosed method mixes the chickpea microcarriers 310, the non-human cells 312, and the cell culture media 318. In some embodiments, the chickpea microcarriers 310 stay in suspension in the cell culture media 318. For example, the chickpea microcarriers 310 float or are otherwise suspended in the cell culture media 318 during mixing.

As indicated above, the disclosed method can mix the non-human cells chickpea microcarriers, and cell culture media at various rates for different lengths of time. For instance, as shown in FIG. 3A, the disclosed method includes an initial time period 304 and a subsequent time period 306. For example, the initial time period 304 occurs when mixing the non-human cells 312, chickpea microcarriers 310, and cell culture media 318 begins. As illustrated, the disclosed method has an initial mixing speed of 50-75 RPMs during the initial time period 304 of 10-20 hours, such as an initial mixing speed of 60 RPM during the initial time period 304 of 16 hours. In alternative embodiments, the disclosed method has an initial mixing speed that does not exceed 75 RPM during the initial time period.

At very small scales, e.g., 30 ml, the RPM may range from 40-75 RPM. However, as someone skilled in the art would understand, when scaling up to larger volumes, RPM will need to be changed and is typically determined empirically at each scale. For example, at larger scales, impellers are often bigger and can rotate more slowly to provide the same level of mixing. In some embodiments, RPM is as slow as possible to ensure adherence of non-human cells to the chickpea microcarriers while avoiding dead zones of no stirring and settling of the chickpea microcarriers while ensuring accurate sampling/testing results. In one or more cases, once the non-human cells attach to the chickpea microcarriers, the disclosed method can raise the RPM to ensure adequate oxygenation and optimal growth without causing excess or detrimental shear.

As further shown in FIG. 3A, the disclosed method changes the initial mixing speed during the subsequent time period 306. Generally, the initial mixing speed during the initial time period 304 allows the non-human cells 312 to contact and adhere to the chickpea microcarriers 310 suspended in the cell culture media 318. After the non-human cells 312 adhere to the chickpea microcarriers 310 during the initial time period 304, the disclosed method increases the initial mixing speed to increase oxygen diffusion, efficiency, etc. For instance, during the subsequent time period 306, the disclosed method increases the initial mixing speed to a subsequent mixing speed. In particular, after an initial time period 304 of 16 hours at the initial mixing speed of 60 RPM, the disclosed method increases the initial mixing speed to the subsequent mixing speed of 100 RPM for a time period of one or more days (e.g., 4 days).

In alternative implementations, the disclosed method may utilize various means of mixing (e.g., agitating) the chickpea microcarriers 310, the non-human cells 312, and the cell culture media 318. To illustrate, the disclosed method may mix the chickpea microcarriers 310, the non-human cells 312, and the cell culture media 318 through shaking, rolling, stirring, blending, rocking, or some combination thereof. Moreover, in some implementations, based on the scale and/or type of the bioreactor 316, the disclosed method can modify and/or scale the mixing rate (e.g., degree of agitation) in order to enable adhesion while avoiding excessive shear according to known methods. For example, the velocity of the cell culture media 318 within the bioreactor 316 should not shear the majority of the non-human cells 312 from the chickpea microcarriers 310.

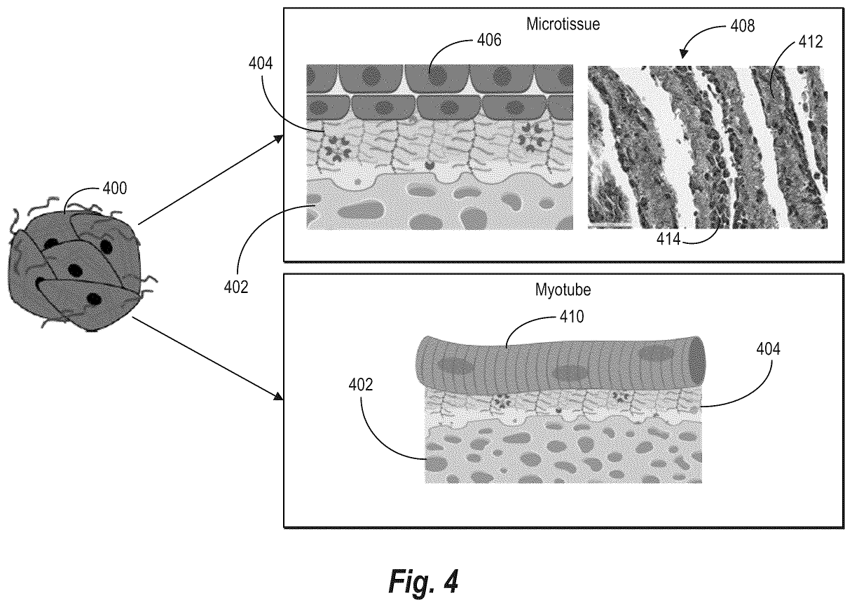

As further shown in FIG. 3A, the disclosed method maintains the subsequent mixing speed for the duration of the subsequent time period 306. Generally, during the subsequent time period 306, the non-human cells 312 adhered to a surface of the chickpea microcarriers grow and differentiate to exhibit a certain phenotype. In some cases, the chickpea microcarriers 310 have a maximum cell carrying capacity. For instance, approximately 3M/ml of non-human cells 312 can attach to 3 mg/mL of the chickpea microcarriers 310. More detail discussing non-human cell growth and differentiation is given in reference to FIG. 4.

As further illustrated in FIG. 3A, the subsequent time period 306 lasts until the disclosed method harvests the textured cell tissue 314. In some embodiments, the subsequent time period 306 lasts up to four days after adding the chickpea microcarriers 310 and the non-human cells 312 to the cell culture media 318. In one or more implementations, the subsequent time period 306 comprises 3-14 days from adding the chickpea microcarriers 310 and the non-human cells 312 to the cell culture media 318. In certain cases, the subsequent time period 306 can exceed 15 days from adding the chickpea microcarriers 310 and the non-human cells 312 to the cell culture media 318. For example, a longer subsequent time period 306 (e.g., greater than 15 days) may allow the non-human cells 312 to differentiate or mature to varying degrees.

In some embodiments, the disclosed method maintains a subsequent mixing speed of 90-110 RPMs (e.g., 100 RPM) from the subsequent time period 306 through a final time period 308. At the final time period 308, the disclosed method harvests textured cell tissue 314 comprising a proliferated population of the non-human cells 312 and the chickpea microcarriers 310. As shown in FIG. 3A, the final time period 308 occurs at the end of the subsequent time period 306 (e.g., four days of mixing). In one or more implementations, the final time period 308 does not exceed 15 days from adding the chickpea microcarriers 310 and the non-human cells 312 to the cell culture media 318.

As discussed above, the disclosed method may add the chickpea microcarriers 310 to the cell culture media 318 after growing the non-human cells 312 to high density. FIG. 3B illustrates an example embodiment of the disclosed method comprising adding chickpea microcarriers 338 after growing the non-human cells in a single cell suspension in accordance with one or more embodiments. As shown in FIG. 3B, the disclosed method includes a seeding phase 322. During the seeding phase 322, the disclosed method adds the non-human cells 332 to cell culture media 334 in suspension in a bioreactor 336.

As indicated above, in some embodiments, the non-human cells 332 are seeded and grown in ACF cell culture media (e.g., SusYL). In other embodiments, the non-human cells 332 are seeded and grown in a growth media with reagents necessary to feed the non-human cells. Alternatively, the cell culture media 334 contains sera derived from animals (e.g., SusYL and 10% FBS).

During the seeding phase 322 depicted in the example embodiment of FIG. 3B, the disclosed method grows the non-human cells 332 in a single cell suspension. In one or more embodiments, the disclosed method grows the non-human cells 332 to a certain density. To illustrate, the disclosed method grows the non-human cells 332 until they reach a relatively high density. For example, the disclosed method grows the non-human cells 332 to a density of 1.5-2.5 million cells per milliliter (e.g., 2 million cells per milliliter). In some embodiments, the seeding density of the non-human cells 332 exceeds 2 million cells per milliliter (e.g., 20,000,000-30,000,000 cells per milliliter). In some embodiments, the disclosed method uses an agitation rate to keep the non-human cells 332 in suspension.

As further shown in the example embodiment of FIG. 3B, after the non-human cells 332 reach a target seeding density (e.g., at least 2 million cells per milliliter), the disclosed method includes the act 324 of adding chickpea microcarriers. In particular, the disclosed method adds chickpea microcarriers 338 to the cell culture media 334 and the non-human cells 332. For example, the disclosed method may add 3 mg/ml of chickpea microcarriers 338 to the cell culture media 334 and the non-human cells 332. As discussed above, the disclosed method may rehydrate the chickpea microcarriers 338 prior to adding them to cell culture media 334 and the non-human cells 332. Alternatively, the disclosed method may add dry chickpea microcarriers 338 to the cell culture media 334 and the non-human cells 332.

As further illustrated by the example embodiment of FIG. 3B, after the disclosed method adds the chickpea microcarriers 338 to the cell culture media 334 and the high-density single cell suspension of non-human cells 332, the disclosed method starts an initial time period 326. As shown in FIG. 3B, the initial time period 326 begins after adding the chickpea microcarriers 338 and lasts for 10-20 hours (e.g., 16 hours). During the initial time period 326, the disclosed method mixes the chickpea microcarriers 338, the non-human cells 332, and the cell culture media 334 within the bioreactor 336. In particular, the disclosed method mixes the chickpea microcarriers 338, the non-human cells 332, and the cell culture media 334 at an initial mixing speed of 50-75 RPMs for 10-20 hours e.g., 60 RPMs for 16 hours). In some embodiments, during the initial time period 326, the disclosed method utilizes a higher initial mixing speed that does not exceed 75 RPM. In one or more implementations, the disclosed method mixes (e.g., agitates) the chickpea microcarriers 338, the non-human cells 332, and the cell culture media 334 through shaking, rolling, stirring, blending, rocking, or some combination thereof.

In some embodiments, mixing the non-human cells 332, the chickpea microcarriers 338, and the cell culture media 334 during the initial time period 326 causes the non-human cells 332 to attach to a surface of the chickpea microcarriers 338. In some implementations, the disclosed method adheres the non-human cells 332 to the chickpea microcarriers 338 by stopping a mixing motion, reducing a mixing rate, adding adherent factors or some combination thereof. For instance, the disclosed method may decrease the initial mixing speed to 30-49 RPMs (e.g., 45 RPMs) during the initial time period 326. Alternatively, the disclosed method may utilize adherent culture to adhere the non-human cells 332 to the chickpea microcarriers 338 and/or to create cell-to-cell adhesions.

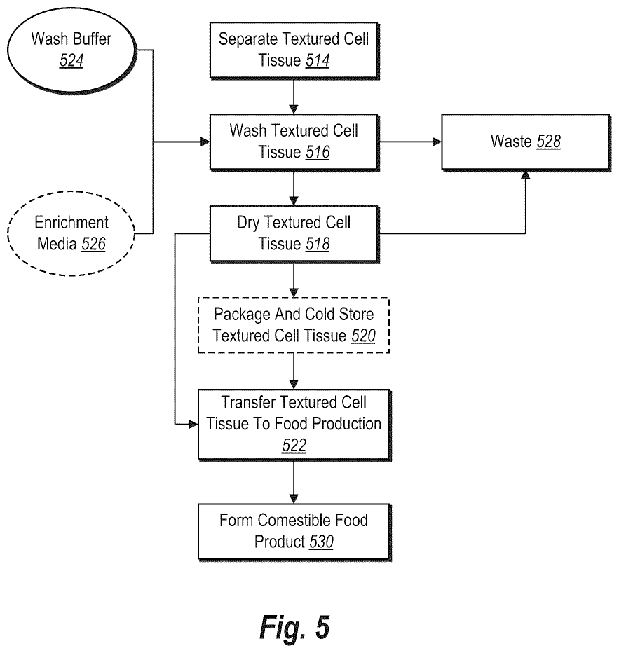

As further illustrated in the example embodiment of FIG. 3B, the disclosed method further comprises a subsequent time period 328 following the initial time period 326. As shown in FIG. 3B, the non-human cells 332 attach to the chickpea microcarriers 338 to form textured cell tissue 340. During the subsequent time period 328, the disclosed method may further grow the non-human cells 332 on the chickpea microcarriers 338. For example, the disclosed method may increase the initial mixing speed to a subsequent mixing speed for 18-36 hours (e.g., 24 hours) prior to harvesting. In some embodiments, the subsequent time period 328 lasts up to four days after adding the chickpea microcarriers 338 to the cell culture media 334 and does not exceed 15 days after adding the chickpea microcarriers 338 to the cell culture media 334. Alternatively, the disclosed method may harvest the textured cell tissue 340 after the initial time period 326 elapses. FIG. 5 provides further detail regarding harvesting the textured cell tissue.

As discussed above, the disclosed method forms a textured cell tissue comprising the chickpea microcarriers and the non-human cells. FIG. 4 depicts the differentiation and types of non-human cells attached to the surface of the chickpea microcarrier in accordance with one or more embodiments.

As shown in FIG. 4, for instance, the textured cell tissue 400 forms microtissue and/or myotubes. As FIG. 4 illustrates, the microtissue comprises chickpea microcarriers 402, an extracellular matrix (ECM) 404, and non-human cells 406. As discussed above, during the initial mixing period, the non-human cells come into contact with and adhere to the surface of the chickpea microcarriers. This attachment allows the non-human cells to contact each other and form cell-to-cell adhesions. In one or more cases, after the non-human cells adhere to each other, they secrete the ECM and form layers of tissue. Thus, in some embodiments, the textured cell tissue 400 comprises the chickpea microcarriers 402, the ECM 404 (e.g., extracellular matrix proteins), and the non-human cells 406.

In certain cases, the non-human cells 406 form more than one layer over a surface of the chickpea microcarrier 402. For example, in one or more implementations, the non-human cells 406 form a first layer over the surface of the chickpea microcarrier 402. In some embodiments, the non-human cells 406 form a second layer over a surface of the chickpea microcarrier 402. As illustrated in FIG. 4, for example, an image of tissue 408 depicts different sections or layers of cell-based meat comprising the non-human cells 414 and collagen 412 (e.g., the ECM). This disclosure depicts the image of tissue 408 as a non-limiting example of the type of different sections or layers of cell-based meat that may form, but such sections and layers of cell-based meat may be different in practice when formed using a chickpea microcarrier.

As mentioned above, the non-human cells 406 form the ECM 404 through secretion. In some embodiments, the ECM 404 helps the non-human cells 406 communicate and differentiate (e.g., turn into more specialized cells with specialized functions). In particular, the non-human cells 406 differentiate to exhibit a certain phenotype characteristic of cells, such as, myoblasts, mesoangioblasts, myofibroblasts, mesenchymal stem cells, hepatocytes, fibroblasts, pericytes, adipocytes, epithelial cells, chondrocytes, osteoblasts, osteoclasts, pluripotent cells, somatic stem cells, endothelial cells, or myogenic stem cells.

FIG. 4 illustrates the textured cell tissue 400 exhibiting a myoblast phenotype. In particular, FIG. 4 shows the textured cell tissue 400 comprising a myotube 410, the ECM 404, and the chickpea microcarrier 402. In general, myotubes are multinucleated muscle fibers that form when myoblasts fuse together through myogenesis. While the myotube 410 may not form in suspension without a microcarrier or the textured cell tissue 400, the chickpea microcarrier 402 within the textured cell tissue 400 creates an environment in which the myotube 410 forms. In particular, as shown in FIG. 4, the disclosed method forms the myotube 410 because the non-human cells adhere to a surface of the chickpea microcarrier 402 and have time to grow and differentiate on the chickpea microcarrier 402. For instance, the non-human cells (e.g., myoblasts) adhered to the chickpea microcarrier had time to align and fuse together to form muscle fibers.

FIG. 5 illustrates an example of harvesting the textured cell tissue in accordance with one or more embodiments. As an overview of FIG. 5, the disclosed method includes separating, washing, and drying textured cell tissue. After such processing, the disclosed method includes packing and transferring the textured cell tissue into a comestible food product (e.g., comestible meat product).

As shown in FIG. 5, for example, the disclosed method comprises an act 514 of separating the textured cell tissue as part of harvesting the textured cell tissue. In some implementations, separating the textured cell tissue comprises separating the cell culture media from the textured cell tissue. In particular, the disclosed method separates the non-human cells adhered to the chickpea microcarriers from the cell culture media. For example, the act 514 comprises draining the cell culture media from the bioreactor.

Additionally, or alternatively, separating the textured cell tissue refers to removing the textured cell tissue from the bioreactor. In some implementations, the act 514 further comprises removing the cells from the bioreactor by passing the cell culture media and textured cell tissue through a filter. For example, transferring the textured cell tissue to a harvest container, such as a jacketed bag filter. The textured cell tissue may be kept in the bioreactor or transferred to a harvest container for additional processing.

As further illustrated in FIG. 5, the disclosed method comprises an act 516 of washing the textured cell tissue. For instance, a wash buffer 524 may be flowed over the textured cell tissue and through the filter. In some embodiments, the wash buffer 524 may be passed through the bag filter holding the textured cell tissue. As indicated by FIG. 5, wash buffer that flows through the textured cell tissue is discarded as waste 528. In some implementations, enrichment media 526 is optionally added to the textured cell tissue in addition to a washing from the wash buffer 524. For example, the enrichment media 526 can be high in antioxidants and include additives that enhance organoleptic properties of the textured cell tissue including taste, appearance, texture, and smell.

FIG. 5 also illustrates an act 518 of drying the textured cell tissue. Generally, the disclosed method can reduce the moisture content of the textured cell tissue in the harvest room. In some embodiments, drying the textured cell tissue comprises pressing the textured cell tissue on a cell press. In other embodiments, the cell tissue may be dried by centrifuging the tissue, filtering the tissue, or other means. The dried textured cell tissue may be transferred to food production as shown by act 522. More specifically, the dried textured cell tissue may directly proceed to food production. In other embodiments, and as shown by act 520, the dried textured cell tissue is packaged and cold stored before proceeding to food production.

FIG. 5 further shows an act 530 of the disclosed method forming a comestible food product. Once the disclosed method reduces the moisture content, the disclosed method can arrange the textured cell tissue to mimic a target meat (e.g., sirloin steak, chicken breast, fish filet, etc.). For example, the disclosed method shapes the textured cell tissue into a cut of comestible meat. For instance, the disclosed method arranges the textured cell tissue to create an internal shape and external shape. Such an external shape is formed with a mold, stamp or some combination thereof.

In some embodiments, the disclosed method aligns portions of the textured cell tissue to exhibit textural variation. For instance, the disclosed method stacks the roughly spherical particles (or other portions) of the textured cell tissue so that the comestible food product has an internal texture and an external texture mimicking the textures of a target meat. In some implementations, the disclosed method forms the comestible food product with textured cell tissue that includes extracellular matrix proteins.

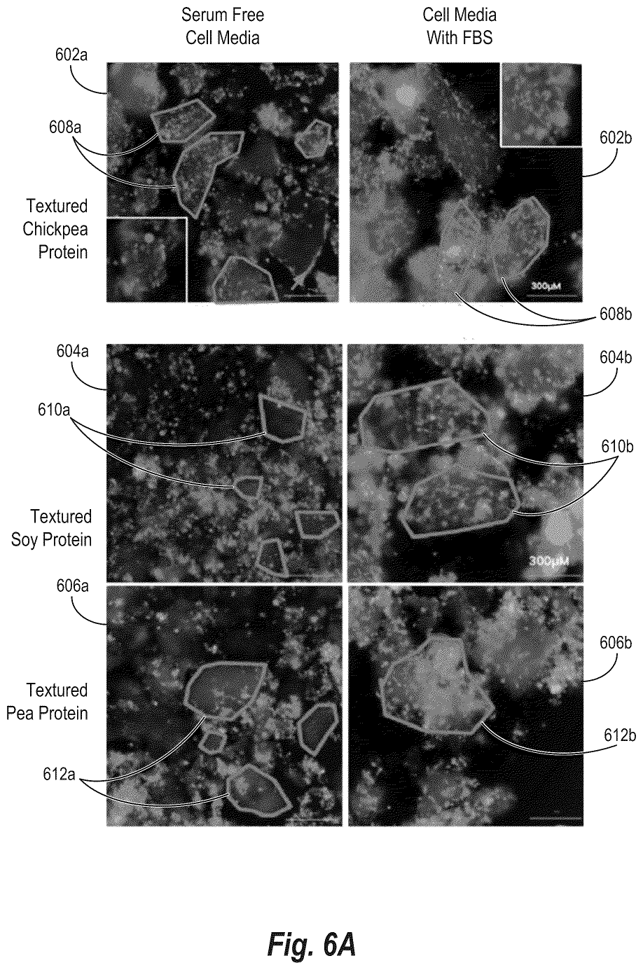

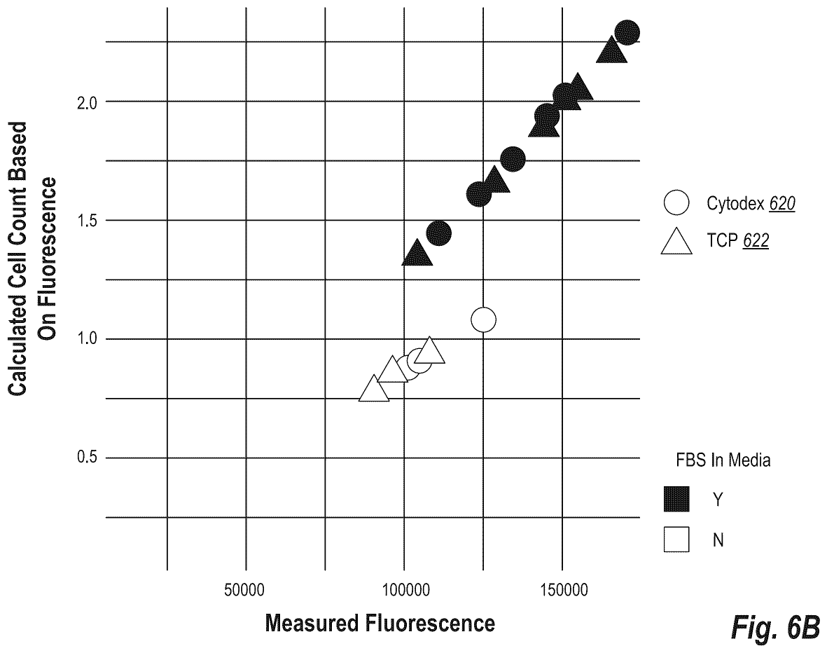

As indicated above, chickpea microcarriers outperform other alternative materials as edible microcarriers. To illustrate, FIGS. 6A-6B compare the degree of attachment between microcarriers made from textured chickpea protein and microcarriers made from other materials in different cell culture media formulations. For example, FIG. 6A compares the degree of attachment between microcarriers made from textured chickpea protein, textured soy protein, and textured pea protein in serum free cell media and cell media with FBS in accordance with one or more embodiments. As indicated by FIG. 6A, half of the well plates were seeded with 250,000 cells/ml in SusYL and the other half with 250,000 cells/ml in SusYL with 10% FBS. A provision of 3 g/L of chickpea microcarriers, soy microcarriers, or pea microcarriers, respectively, were added to the well plates containing SusYL and SusYL with 10% FBS. Additionally, a green fluorescent protein (“GFP”) gene was added to the non-human cells to help capture images of the non-human cells adhering to the microcarriers under a microscope. Each well plate had an initial mixing speed of 60 RPM with 19 mm orbital diameter (e.g., throws) during the initial time period of 16 hours. After 16 hours, the initial mixing speed increased to a subsequent mixing speed of 100 RPM during the subsequent time period. After mixing the well plates at 100 RPM for a total of four days, the textured cell tissue samples were imaged. For example, in some embodiments, prior to imaging, the textured cell tissue is filtered with a cell strainer to wash the cell culture media off the cells. In some embodiments, during imaging, a microscope images the cells at 10× magnification with GFP. In one or more embodiments, the microscope takes images at a different magnification or uses other labeling techniques on the non-human cells, as known in the art. In certain cases, the texture of the harvested textured cell tissue may be analyzed.

As a skilled artisan would understand, however, when scaling up to larger volumes, the mixing rate or speed (e.g., RPM) will change and is typically determined empirically at each scale for a larger orbital diameter. For example, at larger scales, impellers are often bigger and can rotate more slowly to provide the same level of mixing. Accordingly, in some cases, each well plate has an initial mixing speed of 25-40 RPMs with relatively larger orbital diameter (e.g., 0.20 m orbital diameter) during the initial time period of 16 hours. After 16 hours, the initial mixing speed increased to 60-80 RPM during the subsequent time period.

As just mentioned, the scale of the orbital diameter correlates to the mixing rate (e.g., RPM). In certain implementations, the mixing rate (e.g., RPM) changes based on the orbital diameter according to the following equation:

$${r}_{2} = \sqrt{( {r}_{1}^{2} \times \frac{{d}_{1}}{{d}_{2}} )} ,$$

where d₁ represents the orbital diameter for the first shaker, d₂ represents the diameter for a second (e.g., different) shaker, r₁ represents the RPM for the first shaker and r₂ represents the RPM for the second (e.g., different) shaker, as described by Mary Kay Bates, Orbital Shaker Benchmarks: Best Practices for Use and Maintenance, ThermoFisher Scientific Application Note (2017), available at https://assets.thermofisher.com/TFS-Assets/LED/Application-Notes/Orbital-Shaker-Benchmarks-Best-Practices-App-Note-ANMAXQBEST.pdf, which is hereby incorporated by reference in its entirety. For instance, the RPM for scaling up from a system utilizing 25 mm orbital diameter at 100 RPM to a system utilizing a 50 mm orbital diameter is 65-72 RPM.

In certain implementations, when scaling up to larger volumes, the mixing rate may be calculated based on reaching a certain suspension state (e.g., just suspended, nearly homogenous suspension, or homogenous suspension) for various chickpea microcarrier concentrations. For example, in some cases, a Zwietering correlation can help predict the just suspended state, where the mixing rate suspends particles (e.g., chickpea microcarriers) in the cell culture media such that the chickpea microcarriers stay in motion and do not settle on the bottom of the suspension bioreactor for more than 1 to 2 seconds. In some embodiments, the just suspended state can be calculated according to the following equation:

$${N}_{js} = {{Sv}^{0.1} [ \frac{g ( {ρ}_{s} - {ρ}_{l} )}{{ρ}_{l}} ]}^{0.45} {X}^{0.13} {d}_{p}^{0.2} {D}^{- 0.85}$$

where Nⱼₛ represents the just suspended speed, S represents the Zwietering Nⱼₛ constant, v represents the kinematic viscosity

$$( \frac{{m}^{2}}{s} ) ,$$

g represents the gravitation constant

$$( \frac{kg}{{m}^{3}} ) ,$$

ρₛ represents the solid density

$$( \frac{{m}^{2}}{s} ) ,$$

ρ₁ represents liquid density (kg/m³), X represents solids loading

$$( ( \frac{kg solids}{kg liquids} ) \times 1 \; 00 ) ,$$

dₚ represents particle diameter (m), and D represents impeller diameter (m). In some cases, a change in the just suspended state Nⱼₛ can be determined based on a change in the impeller diameter (D), as described in Determining Agitation Requirements for Microcarrier Processes: Method Development Using the Mobius® 50 L Single-Use Bioreactor, Millipore Sigma Application Note (2018), available at https://www.sigmaaldrich.com/deepweb/assets/sigmaaldrich/marketing/global/documents/359/769/mobius-cellready-50l-appnote-an4462en-ms.pdf, which is hereby incorporated by reference in its entirety. In one or more implementations, the mixing rate may be based on a nearly homogenous suspension, where a small population of microcarriers settled on the bottom of the suspension bioreactor nears zero. In certain embodiments, the mixing rate may be based on maintaining a completely homogenous suspension.

As shown in FIG. 6A, the textured chickpea protein in serum free cell media 602a has a higher rate of attachment than the texture soy protein in serum free media 604a and the textured pea protein in serum free media 606a. In particular, the bordered areas 608a representing the chickpea microcarriers show cell attachment. By contrast, the bordered areas representing soy microcarriers 610a and the bordered areas representing pea microcarriers 612a have little to no cell attachment.

As further illustrated in FIG. 6A, the textured chickpea protein 602b, textured soy protein 604b, and textured pea protein 606b all had cell attachment in cell culture media with FBS. As indicated in FIG. 6A, the bordered areas for chickpea microcarriers in cell culture media with FBS 608b, the bordered areas for soy microcarriers in cell culture media with FBS 610b, and the bordered areas for pea microcarriers in cell culture media with FBS 612b all had cell attachment. This is an expected result because animal serum in cell culture media encourages attachment. But the comparison of serum-free and serum-based cell culture media illustrates how chickpea microcarriers can flexibly attach or adhere cells in both serum-free media and serum-based media unlike other edible material options for microcarriers.

FIG. 6B compares the degree of attachment between microcarriers made of textured chickpea protein and positively charged, cross linked dextran (e.g., Cytodex 1) in serum free cell culture media conditions and cell culture media containing FBS in accordance with one or more embodiments. Generally, positively charged, cross linked dextran (“Cytodex”) is a microcarrier used extensively in pharmaceuticals to produce proteins, antibodies, and vaccines. As illustrated in FIG. 6B, the rate of adherence for non-human cells to the microcarriers comprising textured chickpea protein (“TCP”) is similar to the rate of adherence for microcarriers comprising Cytodex.

Regarding the data in FIG. 6B, the microcarriers comprising TCP 622 and the microcarriers comprising Cytodex 620 were seeded with non-human cells in a first set of well plates with cell culture media containing FBS and a second set of well plates containing FBS free cell culture media. After one day of culture in the well plates, the non-human cells in suspension and the non-human cells attached to a surface of the microcarriers were separated by passing the non-human cells and microcarriers through a 70 μm filter. The textured cell mass that did not pass through the filter was washed off into a new well and 500 μL of volume was moved to a 24 well read plate in technical triplicate. 50 μL of AlamarBlue reagent was added to the wells of the read plate, and the 24 well read plate was placed in an incubator for one hour. After one hour, the 24 well read plate was read by a synergy plate reader. Linear models were calculated to relate the AlamarBlue readouts to ViCell readouts (e.g., readouts that measure cell viability, cell concentration and cell growth rate) using data from the suspension cell reads. Utilizing the linear model, the predicted cell counts were calculated for cells on the TCP and Cytodex microcarriers. As illustrated in FIG. 6B, the non-human cells attached to the TCP 622 and the Cytodex 620 at similar rates in cell culture media containing FBS and in cell culture media not containing FBS.

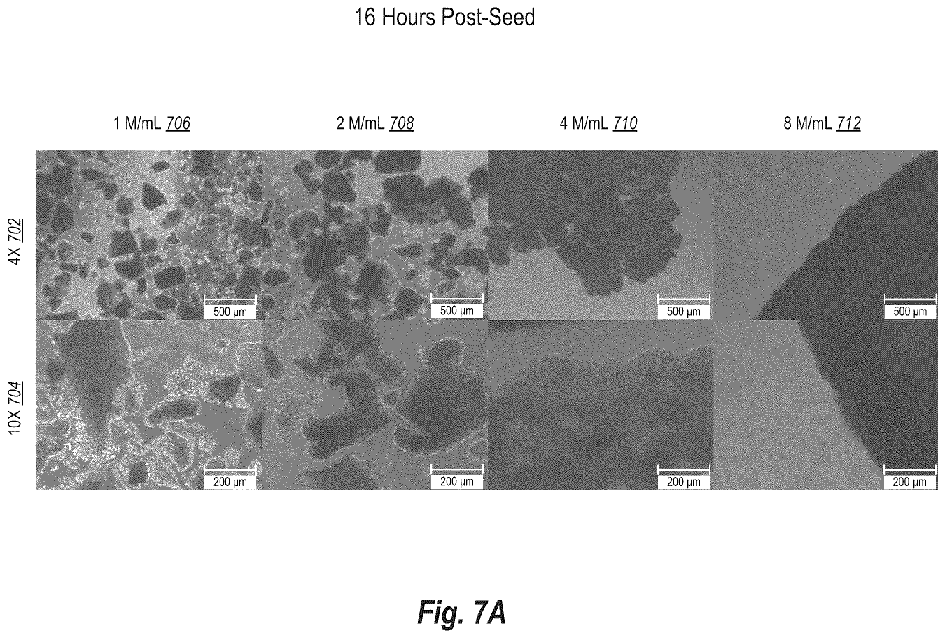

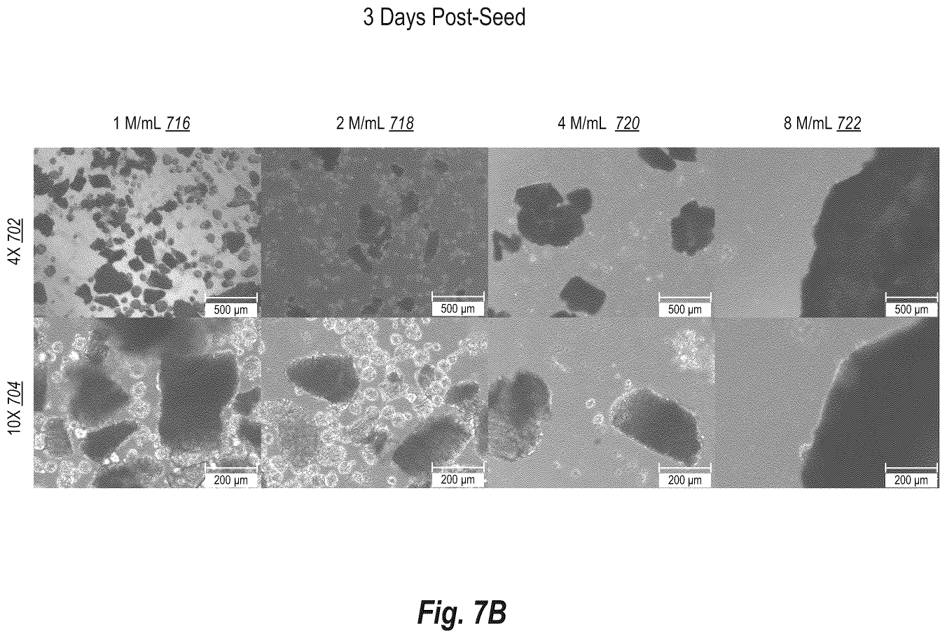

As described above, the disclosed methods can utilize various seeding densities for seeding non-human cells on chickpea microcarriers. FIGS. 7A-7B illustrate various seeding densities of non-human cells at different time periods in accordance with one or more embodiments. In particular, FIG. 7A shows images of non-human cells that were captured in phase contrast under a first magnification 702 of 4× and a second magnification 704 of 10×. The images likewise depict non-human cells that have been seeded at various seeding densities 16 hours post-seeding with chickpea microcarriers (e.g., using a seeding density of 3 mg/mL of textured chickpea protein microcarriers and other various seeding densities). For example, FIG. 7A shows the non-human cells that have been seeded in or through four wells with seeding densities at 1 M/mL 706, 2 M/mL 708, 4 M/mL 710, and 8M/mL 712, where M/mL represents the number of cells in the millions per milliliter of cell culture media (e.g., ACF SusYL).

As further shown in FIG. 7A, after 16 hours, the seeding density of 1 M/mL 706 resulted in several small clumps of non-human cells. Moreover, as FIG. 7A illustrates, increasing the seeding density resulted in larger clumps of the non-human cells. For instance, the clumps of non-human cells at a seeding density of 2M/mL 708 are bigger than the clumps of non-human cells at a seeding density of 1 M/mL 706. As further shown in FIG. 7A, at seeding densities of 4 M/mL 710 and 8 M/mL 712, the non-human cells gather into sheets.

FIG. 7B shows the non-human cells that were initially seeded within or through the same four wells but now three days post-seeding. As indicated in FIG. 7B, at three days post-seeding, the non-human cells formed smaller clumps at all of the seeding densities of 1 M/mL 716, 2 M/mL 718, and 4 M/mL 720 than the seeding density of 8 M/mL 722. As further shown in FIG. 7B, while the non-human cells formed smaller clumps at all conditions, the size of the clumps of the non-human cells at the seeding density of 8M/mL 722 are much larger than the other conditions or seeding densities.

In some cases, too high of a seeding density can inhibit the growth (e.g., proliferative capacity) of the non-human cells by forming the non-human cells into thick clumps or tissue that can induce necrosis. To prevent inducing necrosis, in some embodiments, the disclosed methods can utilize a seeding density that supports proliferation of the non-human cells without inhibiting the growth of the non-human cells. For example, in one or more embodiments, the seeding density of the non-human cells can be between 1M/mL and 4 M/mL. In some embodiments, based on the scale of production, mixing speeds, length of the initial time period, and/or length of the subsequent time period, the disclosed methods can increase or decrease the seeding density of the non-human cells as to enable the proliferation of the non-human cells.

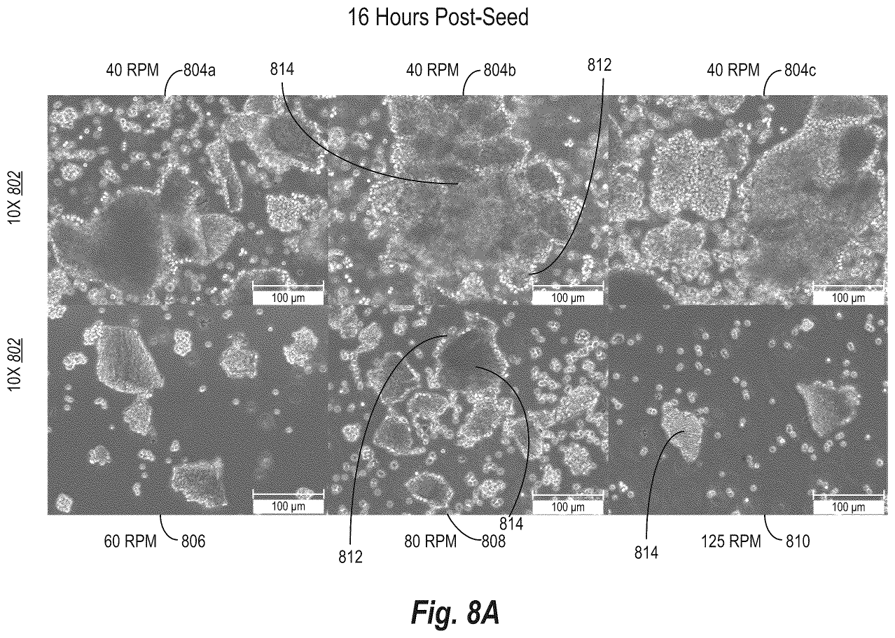

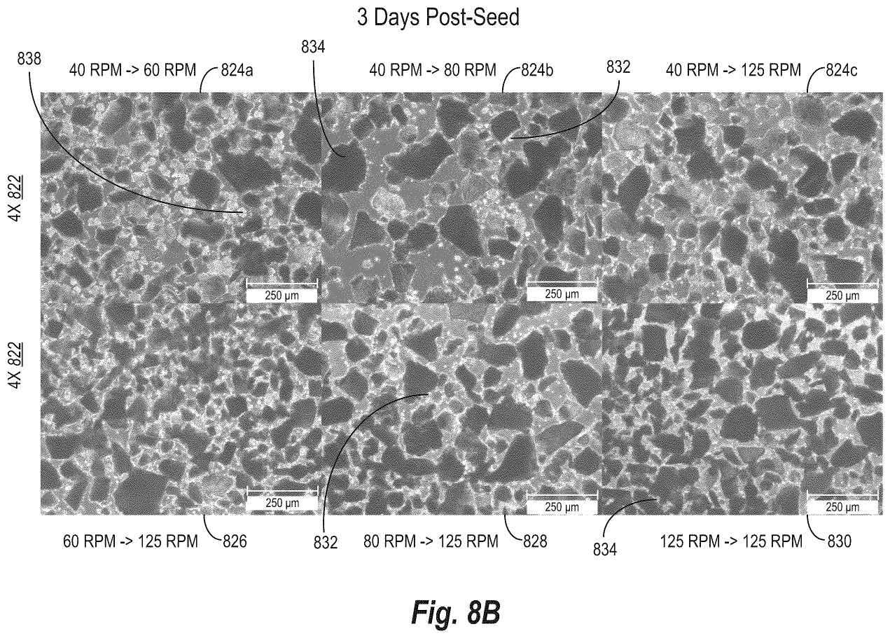

As described above with respect to FIGS. 3A-3B, the disclosed methods can include mixing non-human cells and the chickpea microcarriers at an initial mixing speed during an initial time period and increasing the initial mixing speed to a subsequent mixing speed during a subsequent time period. FIGS. 8A-8B show images of textured cell tissue comprising non-human cells adhered to chickpea microcarriers at different mixing speeds during an initial time period and subsequent time period in accordance with one or more embodiments. In particular, FIG. 8A provides images of the non-human cells 812, where the disclosed method mixes the non-human cells 812 and the chickpea microcarriers 814 at various initial mixing speeds during an initial time period. For example, the disclosed method includes combining 3.88 M/mL of the non-human cells 812 in ACF cell culture media (e.g., SusYL+/−25 mg/L choline chloride) with 3 mg/ml of the chickpea microcarriers 814 in a 125 mL flask to a volume of 25 mL and stirring the non-human cells 812 and the chickpea microcarriers 814 for 16 hours at a 25 mm orbital diameter (e.g., throw).

FIG. 8A includes the images depicting the adherence of the non-human cells 812 to the chickpea microcarriers 814 at the initial mixing speeds of 40 RPM, 60 RPM, 80 RPM, and 125 RPM during the initial time period of 16 hours post-seed. In particular, FIG. 8A includes images 804a, 804b, and 804c showing the non-human cells 812 adhering to the chickpea microcarriers 814 during the initial time period at an initial mixing rate of 40 RPM; an image 806 of the non-human cells 812 adhering to the chickpea microcarriers 814 during the initial time period at an initial mixing rate of 60 RPM; an image 808 of the non-human cells 812 adhering to the chickpea microcarriers 814 during the initial time period at an initial mixing rate of 80 RPM; and an image 810 of the non-human cells 812 adhering to the chickpea microcarriers 814 during the initial time period at an initial mixing rate of 125 RPM. As indicated in FIG. 8A, the images 804a, 804b, and 804c, 806, 808, and 810 were captured under microscope at a magnification 802 of 10×.

In FIG. 8A, the images 804a-c for the initial mixing speed of 40 RPM, and a filtered cell count of the non-human cells 812 at each initial mixing speed, showed that the initial mixing speed of 40 RPM resulted in the largest number of the non-human cells 812 adhering to the chickpea microcarriers 814, followed by such adherence depicted by the image 806 for the initial mixing speed of 60 RPM. For example, the image 806, along with the filtered cell count of the non-human cells 812, shows fewer of the non-human cells 812 attaching to the chickpea microcarriers 814 during the initial time period with an initial mixing speed of 60 RPM relative to the number of the non-human cells 812 attaching to the chickpea microcarriers 814 during the initial time period that utilized the initial mixing speed of 40 RPM. Moreover, the filtered cell count indicated that the initial mixing speed of 80 RPM resulted in fewer of the non-human cells 812 attaching to the chickpea microcarriers 814 during the initial time period when compared to the initial mixing speed of 60 RPM. Finally, the filtered cell count and the image 810 indicate that the initial mixing speed of 125 RPM resulted in the fewest number of the non-human cells 812 attaching to the chickpea microcarriers 814.

As just mentioned, the initial mixing speed of 40 RPM resulted in the highest number of the non-human cells 812 attaching to the chickpea microcarriers 814. As shown in the images 804a-c, 806, 808, and 810 in FIG. 8A, the smaller white spheres depict examples of the non-human cells 812, and the larger darker shapes depict examples of the chickpea microcarriers 814. Indeed, the images 804a-c, 806, 808, and 810 in FIG. 8A support the filtered cell count findings by showing several of the non-human cells 812 adhering to the chickpea microcarriers 814 in the images 804a-c. Indeed, the formation of a scalloped edge on the chickpea microcarriers 814 shown in the images 804a-c, 806, and 808 indicates attachment of the non-human cells 812 to the chickpea microcarriers 814.

As indicated by FIG. 8A, depending on the magnification and/or focal plane of a given image depicting the non-human cells and chickpea microcarriers, the given image may show a cross-section of the chickpea microcarriers and/or the surface of the chickpea microcarriers. For example, the given image shows the cross-section of the chickpea microcarriers when the center of the chickpea microcarriers is dark and surrounded by a bright scalloped or cobblestone-like border comprising the non-human cells. Indeed, in the images 804a-c, 806, 808, and 810 shown in FIG. 8A, the chickpea microcarriers 814 are more fully encompassed by the non-human cells 812 in three-dimensional space than would appear in the images 804a-c, 806, 808, and 810 shown in FIG. 8A. Relatedly, based on the magnification and/or focal plane, the given image can show the surface of the chickpea microcarriers encompassed by the non-human cells. In some embodiments, a given image can show the surface of the chickpea microcarriers encompassed by the non-human cells. For example, images in which the surface of the chickpea microcarriers appear to be covered with white spheres in a cobblestone-like pattern indicates that the given image is capturing the surface of the chickpea microcarriers encompassed by the non-human cells.

As FIG. 8A further illustrates, the image 806 and the image 808 show the non-human cells 812 adhering to the chickpea microcarriers 814 at the initial mixing speed of 60 RPM and the initial mixing speed of 80 RPM, respectively, during the initial time period (e.g., 16 hours). As further indicated in FIG. 8A, the image 810 shows relatively less adherence of the non-human cells 812 to the chickpea microcarriers 814 when mixed at the initial mixing speed of 125 RPM during the initial time period. For example, the sharp edges of the chickpea microcarriers 814 depicted in or indicated by the image 810 for an initial mixing speed of 125 RPM show that the non-human cells 812 did not attach to the chickpea microcarriers 814 to the same degree as the chickpea microcarriers 814 mixed at the initial mixing speeds of 40 RPM, 60 RPM, and 80 RPM. In some cases, the relatively higher initial mixing speeds (e.g., 125 RPM) in the experimental conditions did not enable the non-human cells 812 to attach to the chickpea microcarriers 814.

A skilled artisan would, of course, understand that the relative effectiveness of a given initial mixing speed on adherence of non-human cells to chickpea microcarriers depends on seeding density, volume of cell culture media, size of bioreactor, method of agitation, and other conditions. Thus, in some embodiments, the disclosed method can utilize an initial mixing speed from a range of initial mixing speeds during the initial time period that allows the non-human cells 812 to adhere to the chickpea microcarriers 814. In some cases, the range of initial mixing speeds can comprise between 40 RPM and 80 RPM.

As further shown in FIG. 8A, the initial mixing speed during the initial time period can generate various sizes of clumps of the non-human cells 812 and the chickpea microcarriers 814. For instance, a relatively slower initial mixing speed (e.g., 40 RPM) can form larger clumps of the non-human cells 812 and the chickpea microcarriers 814. For example, as shown in images 804a-c, the initial mixing speed of 40 RPM resulted in larger clumps of the non-human cells 812 adhered to the chickpea microcarriers 814 when compared with the images 806, 808, and 810 of the clumps of the non-human cells 812 adhered to the chickpea microcarriers 814 at the initial mixing speeds of 60 RPM, 80 RPM, and 125 RPM, respectively.

Indeed, in some cases, the disclosed methods can utilize an initial mixing speed that generates clumps of the non-human cells 812 and the chickpea microcarriers 814 within a clump size range. In some embodiments, for instance, the disclosed method can utilize an initial mixing speed that generates clumps of the non-human cells 812 adhered to the chickpea microcarriers 814 with a clump size ranging between 75 micrometers and 125 micrometers. Relatedly, the disclosed method can utilize the initial mixing speed so that the diameter, width, and/or length of the clumps of the non-human cells 812 adhered to the chickpea microcarriers 814 falls between 75 micrometers and 125 micrometers.

As just discussed, FIG. 8A illustrates the effects of an initial mixing speed on the adherence of the non-human cells to the chickpea microcarriers during an initial time period. By contrast, FIG. 8B illustrates the adherence of non-human cells to chickpea microcarriers after increasing the initial mixing speed of an initial time period from 0 to 16 hours post-seeding to a subsequent speed of a subsequent time period of 16 hours to 3 days post-seeding in accordance with one or more embodiments. FIG. 8B shows images 824a-c, 826, 828, and 830 of the non-human cells 832 and the chickpea microcarriers 834 captured three days post-seeding under microscope imaged at a magnification 822 of 4× after increasing the initial mixing speeds of 40 RPM, 60 RPM, 80 RPM, and 125 RPM to subsequent mixing speeds.

As shown in FIG. 8B, the image 824a shows the effects of increasing an initial mixing speed of 40 RPM to a subsequent mixing speed of 60 RPM; the image 824b shows the effects of increasing an initial mixing speed of 40 RPM to a subsequent mixing speed of 80 RPM; the image 824c shows the effects of increasing an initial mixing speed of 40 RPM to a subsequent mixing speed of 125 RPM; the image 826 shows the effects of increasing an initial mixing speed of 60 RPM to a subsequent mixing speed of 125 RPM; and the image 828 shows the effects of increasing an initial mixing speed of 40 RPM to a subsequent mixing speed of 125 RPM. By contrast, the image 830 shows the effects of maintaining (i.e., not increasing) an initial mixing speed of 125 RPM at a subsequent mixing speed of 125 RPM.

As further indicated by FIG. 8B, the image 824a shows that increasing the initial mixing speed from 40 RPM to the subsequent speed of 60 RPM during the subsequent time period allowed the non-human cells 832 to stay adhered to the chickpea microcarriers 834 while forming smaller aggregates 838 of the non-human cells 832. Note, however, that test limitations, including focal length, result in no indication of whether the smaller aggregates 838 comprise just the non-human cells 832 attached to each other or comprise the non-human cells 832 adhered to the chickpea microcarriers 834. Similarly, as shown in FIG. 8B, the image 824b shows that the non-human cells 832 remained adhered to the chickpea microcarriers 834 while increasing the initial mixing speed from 40 RPM to the subsequent mixing speed of 80 RPM. Moreover, the image 824c also shows that the non-human cells 832 remained adhered to the chickpea microcarriers 834 while increasing the initial mixing speed from 40 RPM to the subsequent mixing speed of 125 RPM during the subsequent time period.

Similarly, as further shown in FIG. 8B, the image 826 shows that the non-human cells 832 remained attached to the chickpea microcarriers 834 when the disclosed method increased the initial mixing speed from 60 RPM to the subsequent mixing speed of 125 RPM. Additionally, the image 828 shows that the non-human cells 832 remained attached to the chickpea microcarriers 834 when the disclosed method increased the initial mixing speed from 80 RPM to the subsequent mixing speed of 125 RPM.

Finally, as depicted in FIG. 8B, the image 830 shows that the experimental condition of maintaining the initial mixing speed of 125 RPM from the initial time period to the subsequent time period, resulted in some of the non-human cells 832 adhering to the chickpea microcarriers 834. As discussed above, this initial mixing speed of the experimental conditions resulted in fewer of the non-human cells 832 adhering to the chickpea microcarriers 834 during the initial time period. Based on these findings, in some embodiments, the disclosed methods can utilize a subsequent mixing speed during the subsequent period that avoids the non-human cells 832 from shearing off of the chickpea microcarriers 834.

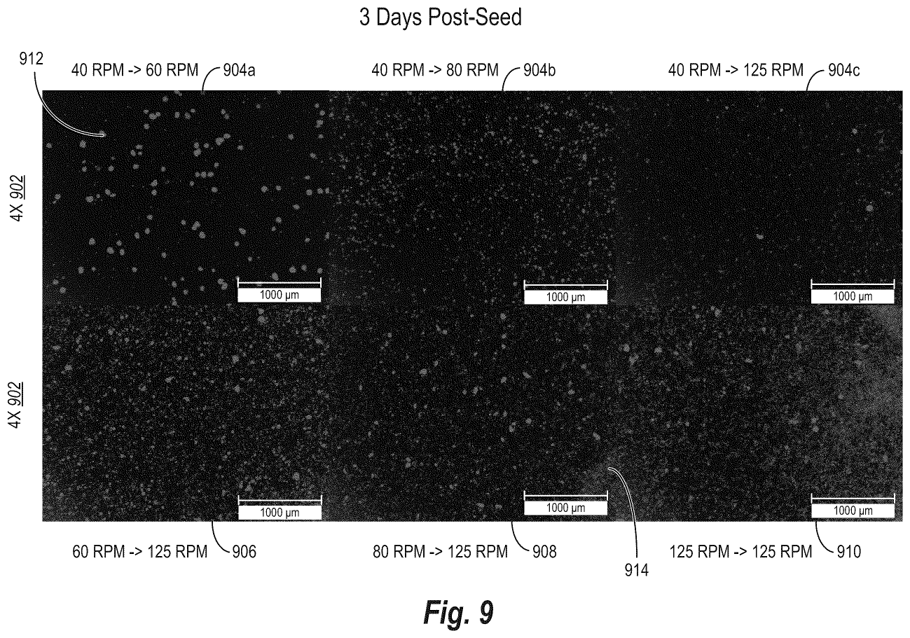

As discussed above, the initial mixing speed and subsequent mixing speed can affect the adherence or attachment of non-human cells to chickpea microcarriers and to other non-human cells. FIG. 9 illustrates how a lower initial mixing speed and subsequent mixing speed can lead to aggregates of non-human cells in accordance with one or more embodiments. In contrast to the images from FIG. 8B depicting non-human cells that did not pass through a filter, however, the images depicted in FIG. 9 show non-human cells that passed through a 70-micrometer filter after increasing an initial mixing speed in an initial time period to a subsequent mixing speed in a subsequent time period.

In particular, FIG. 9 shows images 904a-904c, 906, and 908 at 4× magnification 902 of non-human cells that passed through a 70-micrometer filter after increasing an initial mixing speed at an initial time period to subsequent mixing speeds at a subsequent time period, as follows: the image 904a shows the effects of increasing an initial mixing speed of 40 RPM to an subsequent mixing speed of 60 RPM; the image 904b shows the effects of increasing an initial mixing speed of 40 RPM to a subsequent mixing speed of 80 RPM; the image 904c shows the effects of increasing an initial mixing speed of 40 RPM to a subsequent mixing speed of 125 RPM; the image 906 shows the effects of increasing an initial mixing speed of 60 RPM to a subsequent mixing speed of 125 RPM; and the image 908 shows the effects of increasing an initial mixing speed of 80 RPM to a subsequent mixing speed of 125 RPM. By contrast, the image 910 shows the effects of maintaining an initial mixing speed of 125 RPM at a subsequent mixing speed of 125 from the initial time period to the subsequent time period.

FIG. 9 further shows aggregates of non-human cells 912 and single non-human cells 914 that passed through the 70-micrometer filter. For example, in FIG. 9, the image 904a shows the presence of the aggregates of non-human cells 912, depicted by the larger blue spheres, where the initial mixing speed of 40 RPM increased to a subsequent mixing speed of 60 RPM during the subsequent mixing period. Moreover, the image 904b shows the presence of the single non-human cells 914 when increasing an initial mixing speed of 40 RPM to a subsequent mixing speed of 80 RPM; the image 904c shows the presence of the single non-human cells 914 when increasing an initial mixing speed of 40 RPM to a subsequent mixing speed of 125 RPM; the image 906 shows the presence of the single non-human cells 914 when increasing an initial mixing speed of 60 RPM to a subsequent mixing speed of 125 RPM; and the image 908 shows the presence of the single non-human cells 914 when increasing an initial mixing speed of 40 RPM to a subsequent mixing speed of 125 RPM. By contrast, the image 910 shows the presence of the single non-human cells 914 when maintaining an initial mixing speed of 125 RPM at a subsequent mixing speed of 125 RPM.

As previously described in FIGS. 8A-8B, under the tested conditions, the experimental data indicated that an initial mixing speed of 40 RPM led to the most cell attachment of non-human cells and formation of smaller aggregates of non-human cells during the initial time period (e.g., 16 hours post-seed). FIG. 9 shows aggregates of non-human cells that passed through the 70-micrometer filter after the subsequent time period (3 days post-seed). In some cases, the aggregates of non-human cells 912 can inhibit adherence and/or growth of the non-human cells on the chickpea microcarriers because the non-human cells adhere to each other instead of adhering to the chickpea microcarriers. Thus, in some instances, the disclosed methods can utilize an initial mixing speed that avoids forming aggregates of non-human cells 912 while facilitating attachment to the chickpea microcarriers. For example, the disclosed methods can utilize an initial mixing speed ranging from 50 RPM to 80 RPM that facilitates or creates conditions suitable for non-human cells to adhere to chickpea microcarriers while limiting formation of aggregates of non-human cells.

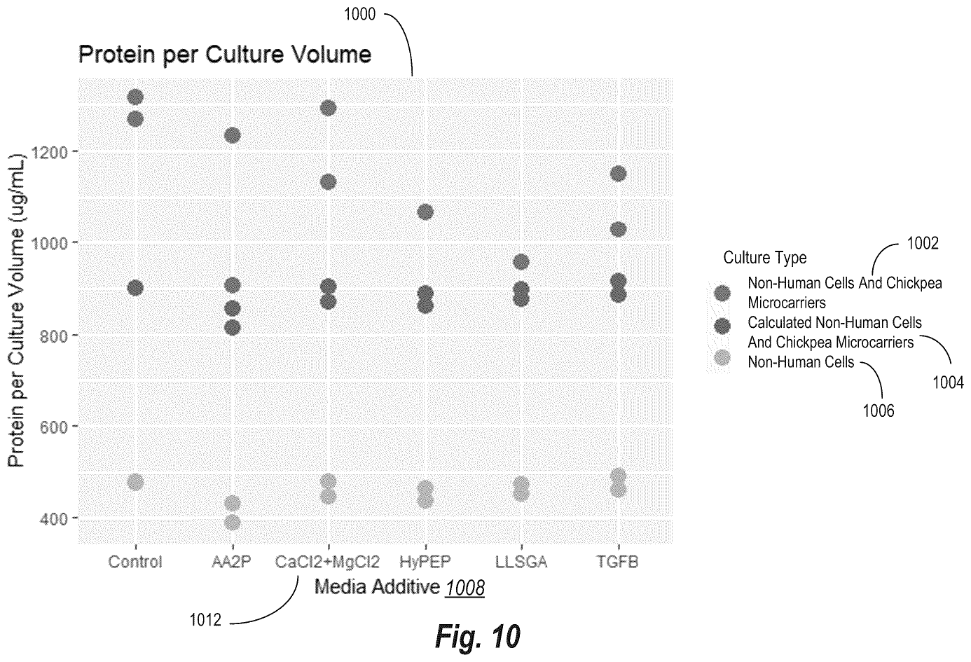

As previously discussed, non-human cells can attach to chickpea microcarriers and form textured cell tissue comprising the non-human cells and the chickpea microcarriers. FIG. 10 shows an amount of protein (e.g., protein per culture volume) of the textured cell tissue with various media additives in accordance with one or more embodiments. In particular, FIG. 10 illustrates a graph 1000 showing the amount of protein (e.g., protein per culture volume) of non-human cells 1006 without chickpea microcarriers, non-human cells cultured with chickpea microcarriers 1002, and calculated amount of protein of the non-human cells and the chickpea microcarriers 1004. As shown in FIG. 10, the graph 1000 includes an x-axis representing the media additive 1008 included in the cell-culture media. For example, the media additive 1008 in the given embodiments included a control of the cell culture media, L-Ascorbic acid 2-phosphate (AA2P), Calcium Chloride and Magnesium Chloride (CaCl₂)+MgCl₂), HyPEP, lipid loaded standard grade albumin (LLSGA), and transforming growth factor beta (TGFB).

As shown in FIG. 10, yellow dots indicate the amount of protein of the non-human cells 1006 grown in culture but without chickpea microcarriers. The green dots represent the calculated amount of protein of the non-human cells and the chickpea microcarriers 1004 where the amount of protein of the non-human cells 1006 in grown in culture is added to the amount of protein (e.g., 420 micrograms/ML) of the chickpea microcarriers combined with the media additive 1008. Accordingly, the green dots indicate an expected amount of protein of the non-human cells and the chickpea microcarriers. The red dots represent the measured amount of protein of the non-human cells grown in culture with the chickpea microcarriers 1002 in the media additive 1008.

As indicated in FIG. 10, the protein per culture volume for the non-human cells adhered to the chickpea microcarriers is higher than the calculated amount of protein of the combined non-human cells and the chickpea microcarriers 1004. Thus, the graph 1000 shows that the non-human cells cultured with the chickpea microcarriers 1002 can produce protein beyond the calculated amount of protein of the non-human cells and the chickpea microcarriers 1004, indicating that the combination of the chickpea microcarriers and the non-human cells synergistically yields greater protein than when chickpea microcarriers and non-human cells are cultured separately. Indeed, the non-human cell growth on chickpea microcarriers results in a surprisingly productive culture where the cells produce more protein than they do when cultured without the chickpea. In some embodiments, the protein of the non-human cells adhered to the chickpea microcarriers can range between 900 micrograms per milliliter of cell culture media and 1500 grams per milliliter of cell culture media.

As just mentioned, the disclosed method tested the effects of combining the media additive 1008 with the non-human cells and the chickpea microcarriers. During testing, the non-human cells and the chickpea microcarriers combined with the magnesium chloride 1012 had less clumping and fewer single non-human cells than another media additive 1008. Thus, indicating that magnesium chloride can aid in the adhesion of the non-human cells to the chickpea microcarriers while diminishing the clumping of the non-human cells to each other.

FIGS. 1-10, the corresponding text, and the examples provide several different systems, methods, techniques, components, and/or devices relating to adding and mixing chickpea microcarriers, non-human cells, and cell culture media, harvesting textured cell tissue from the cell culture media, and forming a comestible food product in accordance with one or more implementations. In addition to the above description, one or more implementations can also be described in terms of flowcharts including acts for accomplishing a particular result. FIGS. 7-10 illustrate such flowcharts of acts. The acts described herein may be repeated or performed in parallel with one another or in parallel with different instances of the same or similar acts.

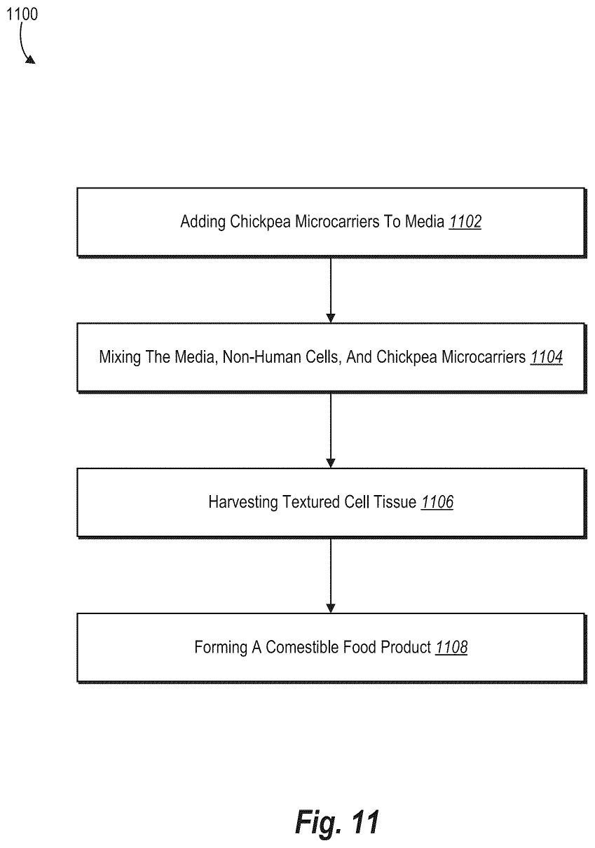

FIG. 11 illustrates a series of acts 1100 comprising an act 1102 of adding chickpea microcarriers to media, and act 1104 of mixing the media, non-human cells, and chickpea microcarriers, and act 1106 of harvesting textured cell tissue, and an act 1108 of forming a comestible food product.

As illustrated in FIG. 11, the series of acts 1100 includes the act 1102 of adding chickpea microcarriers to media. In particular, the act 1102 comprises adding chickpea microcarriers comprising textured chickpea protein to cell culture media.

The series of acts 1100 illustrated in FIG. 11 also includes the act 1104 of mixing the media, non-human cells, and chickpea microcarriers. In particular, the act 1104 comprises mixing, within the cell culture media, non-human cells with the chickpea microcarriers.

As further illustrated in FIG. 11, the series of acts 1100 includes the act 1106 of harvesting textured cell tissue. In particular, the act 1106 comprises harvesting textured cell tissue comprising the non-human cells adhered to the chickpea microcarriers.

FIG. 11 further illustrates the act 1108 of forming a comestible food product. In particular, the act 1108 comprises forming the textured cell tissue into a comestible food product.

In some embodiments, the series of acts 1100 further comprises an act where the cell culture media comprises animal serum-free media. In one or more embodiments, the series of acts also includes an act where the non-human cells differentiate to exhibit a phenotype characteristic of cells comprising myoblasts, mesoangioblasts, myofibroblasts, mesenchymal stem cells, hepatocytes, fibroblasts, pericytes, adipocytes, epithelial cells, chondrocytes, osteoblasts, osteoclasts, pluripotent cells, somatic stem cells, endothelial cells, or myogenic stem cells prior to harvesting.

In certain embodiments, the series of acts 1100 includes an act where the textured cell tissue comprises the non-human cells forming at least one layer over a surface of a chickpea microcarrier of the chickpea microcarriers. In some embodiments, the series of acts 1100 includes an act where the textured cell tissue comprises the non-human cells forming at least one myotube a chickpea microcarrier of the chickpea microcarriers.

In particular embodiments, the series of acts 1100 further comprises mixing the cell culture media, the non-human cells, and the chickpea microcarriers for a time period of no more than 15 days. In one or more embodiments, the series of acts 1100 includes an act where the chickpea microcarriers comprise discrete granules. In some embodiments, the series of acts 1100 further comprises an act where an individual chickpea microcarrier of the chickpea microcarriers does not exceed 150 microns in diameter.

In one or more embodiments, the series of acts 1100 further comprises an act where the chickpea microcarriers suspend in the cell culture media. In some embodiments, the series of acts 1100 further comprises an act where the chickpea microcarriers are hydrated in a separate vessel with media or dried by freeze-drying prior to mixing.

In one or more cases, the series of acts 1100 includes an act where the range of initial mixing speeds comprises 40 RPM to 80 RPM. In one or more implementations the series of acts 1100 includes an act of adding the non-human cells to the cell culture media with a seeding density of 1 million non-human cells per milliliter of the cell culture media to 4 million non-human cells per milliliter of the cell culture media. In particular embodiments, the series of acts 1100 further comprises mixing the non-human cells with the chickpea microcarriers at an initial mixing speed within a range of initial mixing speeds and increasing the initial mixing speed after a time period in which the non-human cells adhere to the chickpea microcarriers. In one or more cases, the series of acts 1100 includes an act where the range of initial mixing speeds comprises 40 rotations per minute (RPM) to 80 RPM.

In certain cases, the series of acts 1100 includes an act where the initial mixing speed within the range of initial mixing speeds supports proliferation of the non-human cells. In one or more instances, the series of acts 1100 includes an act where the initial mixing speed generates clumps of one or more non-human cells and one or more chickpea microcarriers within a threshold range of clump sizes. In some embodiments, the series of acts 1100 includes an act where the threshold range of clump sizes of one or more non-human cells and one or more chickpea microcarriers comprises between 75 micrometers and 125 micrometers. In some cases, the series of acts 1100 includes an act where the initial mixing speed within the range of initial mixing speeds causes the non-human cells to adhere to the chickpea microcarriers while limiting formation of aggregates of the non-human cells.

In certain embodiments, the series of acts 1100 further comprises an act where mixing the non-human cells with the cell culture media comprises: causing the non-human cells to adhere to the chickpea microcarriers by stopping a mixing motion, reducing a mix rate, or adding adherent factors.

In one or more embodiments, the series of acts 1100 further comprises an act where the initial mixing speed does not generate dead zones and suspends the chickpea microcarriers during an initial time period. In some embodiments, the series of acts 1100 includes an act where the initial mixing speed of the initial time period increases to a subsequent mixing speed within a range of subsequent mixing speeds that sufficiently mixes the non-human cells, the chickpea microcarriers, and cell culture media during a subsequent time period. In one or more embodiments, the series of acts 1100 includes an act where the range of subsequent mixing speeds comprises 60 rotations per minute (RPM) to 125 RPM.

In particular embodiments, the series of acts 1100 comprises an act where the textured cell tissue comprises the chickpea microcarriers, extracellular matrix proteins, and the non-human cells. In certain embodiments, the series of acts 1100 further comprises concurrently adding the non-human cells and the chickpea microcarriers to the cell culture media.

In one or more embodiments, the series of acts 1100 also comprises adding the non-human cells to the cell culture media, growing the non-human cells in the cell culture media, and adding the chickpea microcarriers to the cell culture media comprising a plurality of grown non-human cells.

In certain embodiments, the series of acts 1100 includes an act of adding the non-human cells to the cell culture media with a seeding density of 250,000 to 20,000,000 non-human cells per milliliter. In some embodiments, the series of acts 1100 includes an act in which harvesting textured cell tissue comprising the non-human cells adhered to the chickpea microcarriers further comprises removing at least a portion of the cell culture media, and reducing moisture content by pressing the textured cell tissue comprising the non-human cells adhered to the chickpea microcarriers. In one or more implementations, the series of acts 1100 includes an act of adding magnesium chloride to non-human cells and chickpea microcarriers within the cell culture media. In some cases, the series of acts 1100 includes an act where protein of the non-human cells adhering to the chickpea microcarriers ranges between 900 micrograms per milliliter and 1500 micrograms per milliliter.

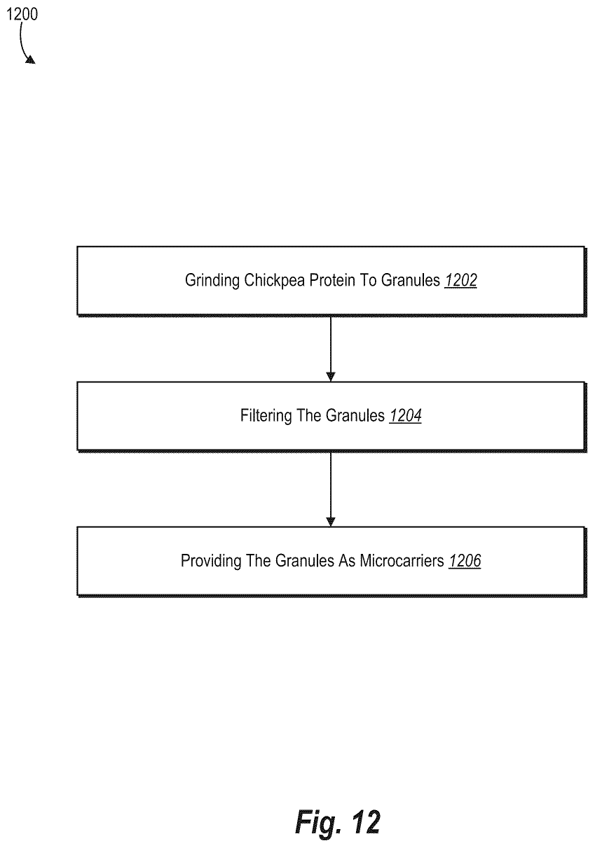

FIG. 12 illustrates a series of acts 1200 comprising an act 1202 of grinding chickpea protein to granules, an act 1204 of filtering the granules, and an act 1206 of providing the granules as microcarriers. For example, in some cases, the act 1206 of providing the granules as microcarriers can include providing the discrete granules of chickpea protein as chickpea microcarriers within cell culture media.

In some embodiments, the series of acts 1200 includes an act in which filtering the discrete granules further comprises utilizing a filter not exceeding 150 microns. In certain embodiments, the series of acts 1200 includes an act where the chickpea protein is dry prior to grinding. In one or more implementations, the series of acts 1200 comprises mixing, within the cell culture media, non-human cells with the chickpea microcarriers, harvesting textured cell tissue comprising the non-human cells adhered to the chickpea microcarriers, and forming the textured cell tissue into a comestible food product.

In some embodiments, the series of acts 1200 includes an act where the discrete granules are hydrated in a separate vessel with media or dried by freeze-drying prior to providing the discrete granules of chickpea protein as the chickpea microcarriers.

As described, this disclosure describes various steps to create a comestible food product and describes various embodiments of a comestible food product. In some embodiments, the comestible food product comprises non-human cells grown from cell culture media; and chickpea microcarriers comprising textured chickpea protein and adhering to the non-human cells. Additionally, in some implementations, the textured cell tissue further comprises a first layer of the non-human cells over a surface of the chickpea microcarriers.

In some embodiments, the textured cell tissue further comprises a second layer of the non-human cells over a surface of the chickpea microcarriers. In one or more embodiments, the non-human cells form a myotube over a surface of the chickpea microcarriers. In certain implementations, the textured cell tissue further comprises extracellular matrix proteins.

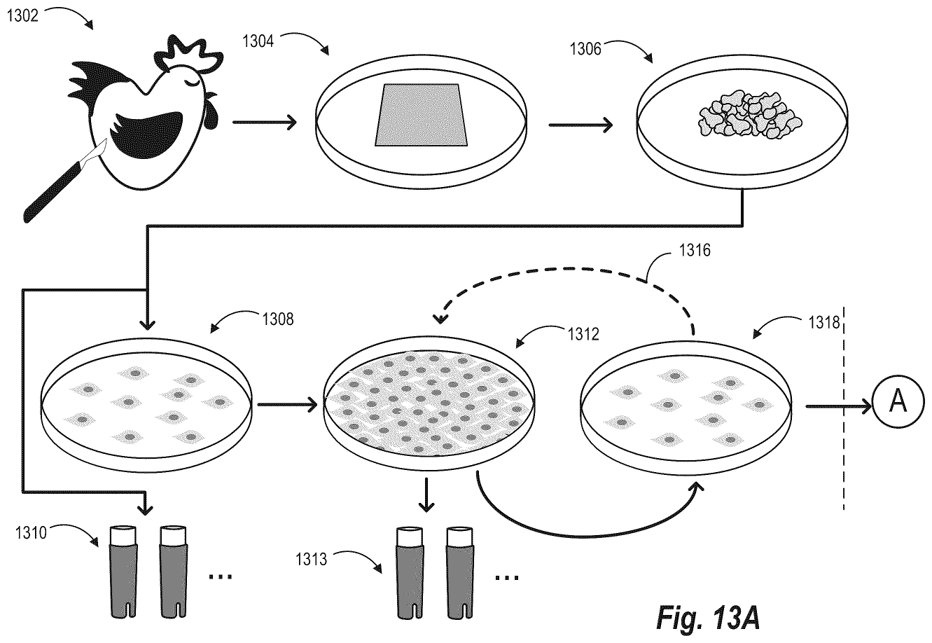

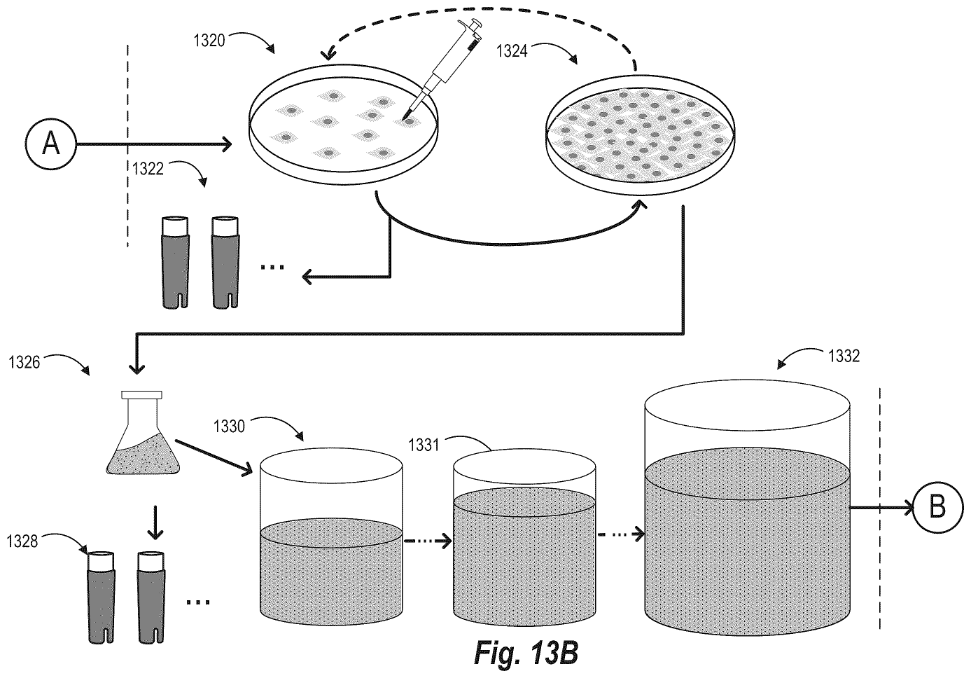

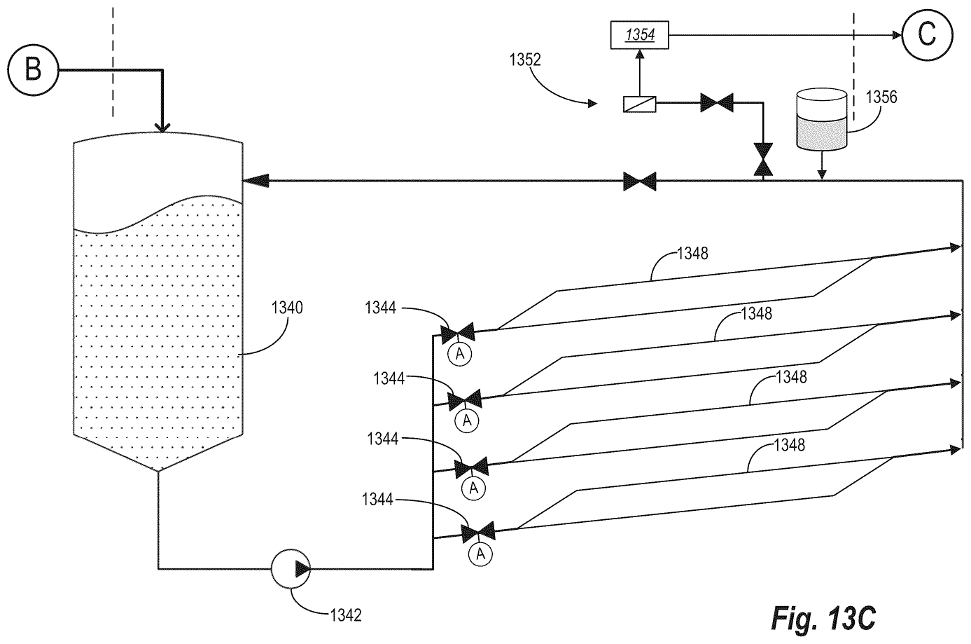

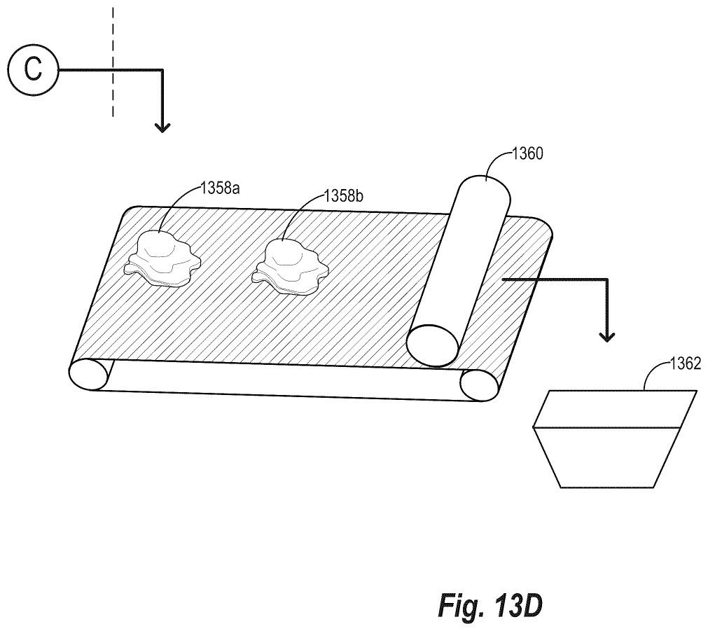

The paragraphs above describe methods for forming a textured cell tissue into a cell-based food product. FIGS. 13A-13D and the following accompanying paragraphs describe procurement of cells and growth of cells into a cell tissue mass in accordance with one or more embodiments. Generally, FIGS. 13A-13D illustrate a process of collecting cells from an animal, growing cells in a favorable environment, banking successful cells, and collecting cells into a cell tissue mass followed by de-wetting and/or other treatments.

As illustrated by step 1302 in FIG. 13A, tissue is collected from a living animal via biopsy. In particular, stem cells, mesenchymal progeny, ectoderm lineage, and/or endoderm lineages can be isolated from the removed tissue. In some implementations of the present disclosure, tissue, such as fat and others, are processed to isolate stem cells, mesenchymal, ectoderm, and/or endoderm progeny or lineage cells. As illustrated, tissue 1304 is removed from an animal. In some examples, the tissue 1304 is removed from a living animal by taking a skin sample from the living animal. For instance, skin or muscle samples may be taken from a chicken, cow, fish, shellfish, or another animal.

Cells may be extracted from the tissue 1304 that was removed from the animal. More specifically, the tissue 1304 is broken down by enzymatic and/or mechanical means. To illustrate, FIG. 13A includes digested tissue 1306 that comprises the cells to be grown in cultivation.

Cells in the digested tissue 1306 may be proliferated under appropriate conditions to begin a primary culture. As illustrated in FIG. 13A, cells 1308 from the digested tissue 1306 are spread on a surface or substrate and proliferated until they reach confluence. As shown in FIG. 13A, in some cases, cells 1312 have reached confluence when they start contacting other cells in the vessel, and/or have occupied all the available surface or substrate.

In some examples, cells are stored and frozen (i.e., banked) at different steps along the cell culture process. Cryopreservation generally comprises freezing cells for preservation and long-term storage. In some implementations, tissue and/or cells are removed from a surface or substrate, centrifuged to remove moisture content, and treated with a protective agent for cryopreservation. For example, as part of cryopreservation, tissues and cells are stored at temperatures at or below −80 C. The protective agent may comprise dimethyl sulfoxide (DMSO) or glycerol.

Cells stored through cryopreservation may be used to replenish working cell stock. For instance, while a portion of the digested tissue 1306 is used as the cells 1308 spread on a surface or substrate, the remaining or excess digested tissue 1306 is transferred to cryovials 1310 for storage. Furthermore, the cells 1312 may be banked once reaching confluence and stored in cryovials 1313.

Once the cells 1312 have reached confluence, or just before the cells 1312 have reached confluence (e.g., occupation of about 80% of the substrate), the disclosed process comprises a series of cell passage steps. During cell passage, the cells 1312 are divided into one or more new culture vessels for continued proliferation. To illustrate, the cells 1312 may be diluted or spread on one or more surfaces or substrates to form the cells 1318. The cells 1318 are then grown 1316 to confluence, or just before confluence.

The cycle of dividing the cells 1312 into the cells 1318 for continued proliferation in new culture vessels may be repeated for a determined number of cycles. Typically, cell lines derived from primary cultures have a finite life span. Passaging the cells allows cells with the highest growth capacity to predominate. In one example, cells are passaged for five cycles to meet a desired genotypic and phenotypic uniformity in the cell population.

In some implementations, the disclosed method comprises immortalizing cells that have been grown and passaged for the determined number of cycles. For instance, the cells 1318 may be immortalized. As shown in FIG. 13B, cells 1320 have demonstrated a preferred growth capacity to proceed to immortalization. To achieve immortalization, the disclosed process transfects the cells 1320 with genes of interest. In one example telomerase reverse transcriptase (TERT) is introduced to the cells 1320. In some embodiments, the cells may be subjected to a selection process as known by those skilled in the art. The cells 1320 may then be passaged for a predetermined set of passaging cycles. In one example passaging cycle, the cells 1320 are grown to (or near) confluence 1324, then they are reseeded in new growth vessels, preserved in vials 1322, or some combination of both. The disclosed process may include any number of passaging cycles to ensure that the cells have reached immortality (e.g., can passage 60+ times without senescing), a target growth capacity, and/or a target quantity for banking. For example, cells may be passaged until they have reached a passage level of 100 (e.g., have been passaged for 100 passaging cycles). In another example, cells are passaged until they reach a population doubling level of 100.

Cells that have reached immortality or a target growth capacity by living through a target passage level may be adapted to suspension culture. In one example, a suspension culture media and agitation of cells in this suspension environment help cells to adapt and start proliferating in the new growth environment. The cells adapted to suspension 1326 may be stored in cryovials 1328 for cryopreservation and banking. Cells in suspension 1326 will begin to proliferate and the process begins a series of dilute and expand steps.

During dilution and expansion, cells are moved from growth vessels into newer, and progressively larger, growth vessels. For example, cells in suspension 1326 may begin in a single tube. The cells will proliferate and increase in cellular density. Once the cells have reached a target cell number (i.e., viable cell density (VCD) at desired volume), they are diluted and moved to a larger growth vessel. Optionally, the cells are banked in cryovials throughout expansion. For example, once cells in suspension reach a maximum VCD, the cells may begin to leave exponential growth due to overcrowding. After reaching a target density, the suspension cells may be transferred to a larger vessel 1330 and diluted with additional media. The dilute-and-expand steps are repeated using progressively larger vessels (e.g., the vessel 1331 and the vessel 1332) and/or progressive dilution until the cells reach a production-ready volume. For example, cells may be production ready at about a 1,000-100,000-liter scale at 5 million cells per mL. The cells may be banked in cryovials at any of the dilution and expansion cycles.

As part of preparing cells to form a comestible food product, the disclosed process comprises growing the cells on microcarriers in a suspension. The cells grown in suspension may remain in the vessel 1332 or may be transferred to a different bioreactor.

FIG. 13C illustrates a bioreactor system comprising a plurality of adherent bioreactors 1348 connecting in parallel to a media vessel 1340. Adherent bioreactors provide an optional finishing step for cells grown in suspension conditions, whereby free-floating cells adhere to substrates and form tissue. The media vessel 1340 holds the cells grown in suspension media. In some implementations, cells from the vessel 1332 are transferred directly to a cell culture media (or just “media”) vessel 1340. In one example, the media vessel 1340 comprises the vessel 1332. As shown, a plurality of valves 1344 is secured to the plurality of adherent bioreactors 1348 to enable individual use and access of each of the adherent bioreactors 1348. For instance, to limit flow to only a first bioreactor of the plurality of adherent bioreactors 1348, the valve 1344 of the first bioreactor is opened while the remaining valves 1344 are closed. Furthermore, the bioreactor system can include a directional valve 1342 for changing between flow directions.

In some implementations, and as illustrated in FIG. 13C, cells (e.g., suspension cells) are prepared by flowing cells suspended in media (e.g., cell culture media) into the plurality of adherent bioreactors 1348. Cells and media that flowed through the adherent bioreactors 1348 are cycled back to the media vessel 1340. The media and cells can be cycled through the adherent bioreactors 1348 until a target adhered cell volume is reached. For instance, in some implementations, the disclosed method comprises measuring a cell density of outflow from the adherent bioreactors 1348 to infer a seeded cell volume.

Prior to optionally finishing the cells of the present disclosure in the adherent bioreactors 1348, the cells are grown in suspension conditions to grow into cell tissue adhering to the microcarriers. Once they have grown to a target density, either according to a learned timing or according to a measured fluctuation in cell metabolism of components such as glucose and oxygen, then the cell tissue is ready for removal or further processing. The removal process of the disclosed method uses filters to separate the cell tissue from the media. The wash buffer 1356 and cell tissue are flowed through a filter 1352 where the cell tissue is collected into one or more cell tissue masses 1354.

As described above and depicted in FIGS. 1 and 3A-3B, for example, this disclosure describes a method of growing non-human cells on chickpea microcarriers suspended in cell culture media as part of forming cell-based food products. In one or more embodiments, however, the disclosed process comprises growing the non-human cells on the chickpea microcarriers in an adherent culture. For example, the non-human cells, chickpea microcarriers, and cell culture media may be transferred for growth on a substrate. For instance, the non-human cells can be transferred from a suspension bioreactor to a plurality of adherent bioreactors. In one or more cases, the chickpea microcarriers are added to the adherent bioreactors. In some embodiments, the adherent bioreactors comprise pipe-based bioreactors attached to a plurality of valves that enable individual use and access of each of the adherent bioreactors. For instance, to limit flow to only a first bioreactor of the plurality of adherent bioreactors, the valve of the first bioreactor is opened while the remaining valves are closed. Furthermore, the bioreactor system can include a directional valve for changing between flow directions.

In some implementations, the non-human cells are prepared by flowing the non-human cells and chickpea microcarriers suspended in cell culture media across substrates in the plurality of adherent bioreactors. More particularly, the non-human cells and chickpea microcarriers from the suspension bioreactor vessel may contact or land on the substrates in the plurality of adherent bioreactors. The non-human cells, chickpea microcarriers, and cell culture media that flowed through the adherent bioreactors are cycled back to the suspension bioreactor vessel. The cell culture media, non-human cells and chickpea microcarriers can be cycled through the adherent bioreactors until a target adherent cell volume is reached. For instance, in some implementations, the disclosed method comprises measuring a cell density of outflow from the adherent bioreactors to infer an adherent cell volume.

The non-human cells grow into adherent textured cell tissue within the adherent bioreactors. Once they have grown to a target volume or quality, either according to a learned timing or according to a measured fluctuation in cell metabolism of components such as glucose and oxygen, then the adherent textured cell tissue is ready for removal. The removal process of the disclosed method uses a high-pressure flow to shear the adherent textured cell tissue comprising the non-human cells and chickpea microcarriers off the substrate surfaces. In one example, wash buffer from a wash tank is flowed across the substrates in the adherent bioreactors. The wash buffer and textured cell tissue mixture are flowed through a filter where the textured cell tissue is collected into one or more cell tissue masses.

The cell tissue masses 1354 may be further processed to adjust moisture content. FIG. 13D illustrates an example apparatus for reducing moisture content in the cells. In particular, FIG. 13D illustrates a pressure apparatus 1360 that compresses the cell tissue masses 1358a and 1358b. While FIG. 13D illustrates a mechanical method for adjusting moisture content of the cell tissue masses 1358a and 1358b, other methods may be used to adjust moisture content. For example, the cell tissue masses 1358a and 1358b may be mixed with a drying agent, vacuum dried, centrifuged, or otherwise dried. A moisture-adjusted-cell tissue mass may be transferred to a container 1362 for additional processing. For example, the cell tissue masses 1358a or 1358b may be removed from the container 1362 to be formed into a cell-based food product.

In accordance with common practice, the various features illustrated in the drawings may not be drawn to scale. The illustrations presented in the present disclosure are not meant to be actual views of any particular apparatus (e.g., device, system, etc.) or method, but are merely idealized representations that are employed to describe various embodiments of the disclosure. Accordingly, the dimensions of the various features may be arbitrarily expanded or reduced for clarity. In addition, some of the drawings may be simplified for clarity. Thus, the drawings may not depict all of the components of a given apparatus (e.g., device) or all operations of a particular method.

Terms used herein and especially in the appended claims (e.g., bodies of the appended claims) are generally intended as “open” terms (e.g., the term “including” should be interpreted as “including, but not limited to,” the term “having” should be interpreted as “having at least,” the term “includes” should be interpreted as “includes, but is not limited to,” etc.).

Additionally, if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” or “one or more of A, B, and C, etc.” is used, in general such a construction is intended to include A alone, B alone, C alone, A and B together, A and C together, B and C together, or A, B, and C together, etc. For example, the use of the term “and/or” is intended to be construed in this manner.

Further, any disjunctive word or phrase presenting two or more alternative terms, whether in the description, claims, or drawings, should be understood to contemplate the possibilities of including one of the terms, either of the terms, or both terms. For example, the phrase “A or B” should be understood to include the possibilities of “A” or “B” or “A and B.”

However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations.

Additionally, the use of the terms “first,” “second,” “third,” etc., are not necessarily used herein to connote a specific order or number of elements. Generally, the terms “first,” “second,” “third,” etc., are used to distinguish between different elements as generic identifiers. Absent a showing that the terms “first,” “second,” “third,” etc., connote a specific order, these terms should not be understood to connote a specific order. Furthermore, absent a showing that the terms “first,” “second,” “third,” etc., connote a specific number of elements, these terms should not be understood to connote a specific number of elements. For example, a first widget may be described as having a first side and a second widget may be described as having a second side. The use of the term “second side” with respect to the second widget may be to distinguish such side of the second widget from the “first side” of the first widget and not to connote that the second widget has two sides.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present disclosure have been described in detail, it should be understood that the various changes, substitutions, and alterations could be made hereto without departing from the spirit and scope of the present disclosure.

The present invention may be embodied in other specific forms without departing from its spirit or essential characteristics. Indeed, the described embodiments are to be considered in all respects only as illustrative and not restrictive. For example, the methods described herein may be performed with less or more steps/acts or the steps/acts may be performed in differing orders. Additionally, the steps/acts described herein may be repeated or performed in parallel to one another or in parallel to different instances of the same or similar steps/acts. The scope of the invention is, therefore, indicated by the appended claims rather than by the foregoing description. All changes that come within the meaning and range of equivalency of the claims are to be embraced within their scope.

1-38. (canceled)

39. A method of forming cell-based food products comprising:
adding chickpea microcarriers comprising textured chickpea protein to cell culture media;
mixing, within the cell culture media, non-human cells with the chickpea microcarriers;
harvesting textured cell tissue comprising the non-human cells adhered to the chickpea microcarriers; and
forming the textured cell tissue into a comestible food product.

40. The method of claim 39, wherein the cell culture media comprises animal serum-free media.

41. The method of claim 39, wherein the non-human cells differentiate to exhibit a phenotype comprising: myoblasts, mesoangioblasts, myofibroblasts, mesenchymal stem cells, hepatocytes, fibroblasts, pericytes, adipocytes, epithelial cells, chondrocytes, osteoblasts, osteoclasts, pluripotent cells, somatic stem cells, endothelial cells, or myogenic stem cells prior to harvesting.

42. The method of claim 39, wherein the chickpea microcarriers comprise discrete granules and are suspended in the cell culture media.

43. The method of claim 39, further comprising: adding the non-human cells to the cell culture media with a seeding density of 1 million non-human cells per milliliter of the cell culture media to 4 million non-human cells per milliliter of the cell culture media.

44. The method of claim 39, further comprising:
mixing the non-human cells with the chickpea microcarriers at an initial mixing speed within a range of initial mixing speeds; and
increasing the initial mixing speed after a time period in which the non-human cells adhere to the chickpea microcarriers.

45. The method of claim 44, wherein the initial mixing speed within the range of initial mixing speeds causes the non-human cells to adhere to the chickpea microcarriers while limiting formation of aggregates of the non-human cells.

46. The method of claim 45, wherein the initial mixing speed within the range of initial mixing speeds supports proliferation of the non-human cells.

47. The method of claim 46, wherein the initial mixing speed generates clumps of one or more non-human cells and one or more chickpea microcarriers within a threshold range of clump sizes.

48. The method of claim 47, wherein the threshold range of clump sizes of one or more non-human cells and one or more chickpea microcarriers comprises between 75 micrometers and 125 micrometers.

49. The method of claim 48, wherein the initial mixing speed does not generate dead zones and suspends the chickpea microcarriers during an initial time period.

50. The method of claim 39, wherein the textured cell tissue comprises the chickpea microcarriers, extracellular matrix proteins, and the non-human cells.

51. The method of claim 39, further comprising:
concurrently adding the non-human cells and the chickpea microcarriers to the cell culture media.

52. The method of claim 39, further comprising:
adding the non-human cells to the cell culture media;
growing the non-human cells in the cell culture media; and
adding the chickpea microcarriers to the cell culture media comprising a plurality of grown non-human cells.

53. The method of claim 39, further comprising adding magnesium chloride to one or more non-human cells and one or more chickpea microcarriers within the cell culture media.

54. The method of claim 39, wherein a protein content of the non-human cells cultured with the chickpea microcarriers is greater than a protein content of the non-human cells cultured separately from the chickpea microcarriers.

55. A comestible food product of textured cell tissue comprising:
non-human cells grown from cell culture media; and
chickpea microcarriers comprising textured chickpea protein and adhering to the non-human cells.

56. The comestible food product of claim 55, wherein the textured cell tissue further comprises a first layer of the non-human cells over a surface of the chickpea microcarriers and a second layer of the non-human cells over the surface of the chickpea microcarriers.

57. The comestible food product of claim 55, wherein the non-human cells form a myotube over a surface of the chickpea microcarriers.

58. The comestible food product of claim 55, wherein the textured cell tissue further comprises extracellular matrix proteins.
